(19) United States
(12) Patent Application Publication
(10) Pub. No.: US 2006/0127400 A1
(43) Pub. Date: Jun. 15, 2006

(54) Activation of regulatory T cells by alpha-melanocyte stimulating hormone

(75) Inventors: Andrew W. Taylor, Waltham, MA (US); Tomomi Nishida, Yokohama (JP)

Correspondence Address: WEINGARTEN, SCHURGIN, GAGNEBIN & LEBOVICI LLP, TEN POST OFFICE SQUARE, BOSTON, MA, 02109, US

(73) Assignee: THE SCHEPENS EYE RESEARCH INSTITUTE, INC.

(21) Appl. No.: 11/346,938

(22) Filed: Feb. 3, 2006

Related U.S. Application Data
(63) Continuation of application No. 09/912,670, filed on Jul. 23, 2001, now abandoned.
Application No. 09/912,670 is a continuation-in-part of application No. PCT/US00/01608, filed on Jan. 21, 2000.
(60) Provisional application No. 60/116,851, filed on Jan. 22, 1999.
(60) Provisional application No. 60/156,788, filed on Sep. 30, 1999.

(51) Int. Cl. A61K 39/395; C12P 21/04; C12N 5/06

(52) U.S. Cl. 424/144.1; 435/338; 435/70.21

(57) ABSTRACT

The invention encompasses a method of down-regulating a T cell-mediated immune response, through activation or T cell receptor (TCR) stimulation of antigen-primed T cells in the presence of alpha-melanocyte stimulating hormone (α-MSH), which may be optionally enhanced by adding transforming growth factor-β2 (TGF-β2) approximately 4-6 hours after the start of the primed T cells' exposure to α-MSH. Activation of the primed T cells may be mediated by presentation of the specific antigen to the primed T cells, or by an anti-TCR antibody or a T cell mitogen. As a result of the α-MSH treatment modulating the T cell activation, antigen-specific, regulatory, CD4+/CD25+ T cells are generated that produce transforming growth factor-β (TGF-β) and can non-specifically down-regulate Th1-mediated inflammatory activities. The method may be used to down-regulate or suppress an autoimmune condition or a graft rejection in a transplant patient. The invention also encompasses a kit for generating regulatory T cell comprising a specific antigen, α-MSH, and optionally, TGF-β2 and/or a T cell culture medium. Also provided are gene therapy treatments for suppressing an autoimmune or graft rejection response, or for re-establishing autotolerance, by introducing genetic material (e.g. nucleic acid) for expressing α-MSH or a receptor-binding portion thereof, into a localized tissue site.

### CROSS-REFERENCE TO RELATED APPLICATIONS

This application is a continuation of U.S. patent application Ser. No. 09/912,671 filed on Jul. 23, 2001, which was a continuation-in-part of and claimed priority from Patent Cooperation Treaty Application No. PCT/US00/01608, filed on Jan. 21, 2000, which application claimed priority from U.S. Provisional Application No. 60/116,851, filed on Jan. 22, 1999, and U.S. Provisional Application No. 60/156,788, filed on Sep. 30, 1999, the whole of which are hereby incorporated by reference herein.

### STATEMENT REGARDING FEDERALLY SPONSORED RESEARCH OR DEVELOPMENT

Part of the work leading to this invention was carried out with United States Government support provided under Grant No. EY10752 from the National Eye Institute of the National Institutes of Health. Therefore, the U.S. Government has certain rights in this invention.

### FIELD OF THE INVENTION

The present invention relates to the regulation of T cell-mediated inflammation.

### BACKGROUND OF THE INVENTION

The induction of a delayed type hypersensitivity (DTH) response is dependent on activation of IFN-γ producing Th1 cells¹. The activation not only requires cognate antigen presenting cells, but also a microenvironment that favors activation and development of the DTH-mediating Th1 cells. To regulate activation of a DTH response, several mechanisms are physiologically employed, such as apoptosis and anergy². In addition, soluble factors in the regional microenvironment of T cell activation can also influence the course of a T cell response³. The presence of specific cytokines can favor expression of specific effector responses while suppressing others⁴⁻⁸. Previously we have found that in the presence of the neuropeptide α-melanocyte stimulating hormone (α-MSH), antigen-activated. Th1 cells were suppressed in their IFN-γ production but continued to proliferate, suggesting that α-MSH may regulate selective effector T cell activities.

The neuropeptide α-MSH is an evolutionarily conserved tridecapeptide derived from the endoproteolytic cleavage of adrenocorticotropic hormone (ACTH) which is in turn a post-translational product of pro-opiomelanocortin hormone (POMC)¹⁰. Initially, α-MSH was described as a pituitary hormone that mediates melanogenesis in amphibians¹¹. In mammals, α-MSH is able to mediate melanogenesis and neurotransmitter activities; however, α-MSH is most potent in functioning as a neuroimmunomodulator¹² α-MSH suppresses inflammation mediated by host defense mechanisms of innate (endotoxin mediated) and of adaptive immunity (T cell mediated). Its anti-inflammatory activity has suggested that α-MSH functions as a necessary physiological regulator of inflammation.

One of the first indications of a link between the immune and nervous systems was in the induction of fever mediated by the systemic effects of IL-1, TNF or endotoxin (Reviewed in (12)). Intracerebroventricular (icv) injection of α-MSH suppresses endotoxin and inflammatory cytokine induced fever. Peripheral injections of α-MSH, although at much higher concentrations then central injections, were also effective in suppressing fever. Injections of anti-α-MSH antibodies icv to neutralize CNS α-MSH activity enhanced IL-1 induced febrile response¹³. In addition, during acute phase responses α-MSH concentration in plasma and in discrete sites of the CNS are elevated^{14,15}. These findings demonstrate that α-MSH antagonizes the CNS response to IL-1, TNF, and endotoxin to regulate the intensity of the febrile response.

Localized peripheral inflammatory responses induced by IL-1, TNF, and endotoxin are also suppressed by α-MSH, regardless if α-MSH is delivered via icv, intravascular, or direct injection to the site¹⁶. Endotoxin and interferon (IFN)-γ activated macrophages cultured with α-MSH are suppressed in generating nitric oxide and in producing TNF and chemokines^{17,18}. Also, α-MSH suppresses in vivo neutrophil migration in response to endotoxin^{18,19}. These findings further indicate that α-MSH antagonizes the activity of inflammatory cytokines and also their synthesis. In addition, α-MSH induces its own production and expression of its receptors on macrophages¹⁷, suggesting that α-MSH can regulate an inflammatory response through autocrine mechanisms. Therefore, various inflammatory mediating events could trigger production of α-MSH that in turn regulates the extent of the inflammatory response. Intravenous injection of α-MSH at the time of applying skin-reactive chemicals suppresses systemic induction and regional expression of contact hypersensitivity²⁰ leading to hapten-specific tolerance²¹. It has recently been found that α-MSH at physiological concentrations induces IL-10 production by antigen presenting cells²². It is possible that α-MSH indirectly promotes tolerance to hapten by inducing IL-10 production by hapten-presenting APC. Therefore, a regulatory cytokine network initiated by α-MSH could suppress both induction and expression of contact hypersensitivity. However, such a regulatory network does not preclude the possibility that α-MSH can directly suppress or affect the responding T cells. That possibility is the focus of the present invention.

Suppression of delayed type hypersensitivity (DTH) is mediated in part by α-MSH within the immune privileged ocular microenvironment^{9,23}. The fluid filling the anterior chamber of mammalian eyes, aqueous humor, contains bioactive α-MSH²³. When α-MSH activity was neutralized, aqueous humor was unable to suppress Th1 activity by activated primed T cells in vitro⁹. Cultures of Th1 cells stimulated with antigen and antigen presenting cells are suppressed in their IFN-γ production by α-MSH, while their proliferation is unaffected⁹. This observation suggests that the signals needed by T cells for IFN-γ production are inhibited by α-MSH, whereas the signals for proliferation are not suppressed. Since both the antigen presenting cells and T cells are affected by α-MSH, when only the primed Th1 cells are pretreated with α-MSH, the production of IFN-γ is profoundly suppressed. However, T cell proliferation again is unaffected⁹. Therefore, these results suggest that T cells are receptive to α-MSH, and are suppressed in mediating inflammatory activity when activated in the presence of α-MSH. The present invention results from characterizing the effects of α-MSH on TCR-stimulated primed T cells, which has shown that the T cells are target cells for α-MSH regulation, and that its suppression of IFN-γ production is due to α-MSH deflecting Th1 cells away from their expected inflammatory response toward a suppressive response.

Over the past 30 years, research into the mechanisms of ocular immune privilege has lead to the understanding that it is an active process mediated in part by the constitutive production of immunosuppressive factors within the ocular microenvironment.^{A1-A3} Immune privilege involves mechanisms that suppress induction of an inflammatory immune response within the eye. As mentioned, this suppression of the induction of immunity to antigen in the eye is mediated in part by immunosuppressive factors found in aqueous humor.^{A3-A7} The constitutive expression of these immunosuppressive factors appears to regulate systemic and regional immune responses to antigen within the ocular microenvironment.

The activation, type, and intensity of an effector T cell response is not limited to antigen sensitivity alone, but also to local immunoregulatory mechanisms to which neurologically derived factors, such as α-MSH, can contribute. This regional regulation insures that the most effective immune defense is mounted in proportion with preserving the unique functionality of the affected tissue. As mentioned, an extreme example of regional immunity is the immune-privileged microenvironment of the ocular anterior chamber.^{B1} Within this microenvironment, delayed type hypersensitivity-mediating T cells are suppressed.^{A9} This suppression is mediated by factors constitutively produced within the anterior chamber.^{B4-B8} Specific neuropeptides are present within the ocular microenvironment that help to maintain the immunosuppression.^{A1} Changes in neuropeptide expression by neurons that innervate ocular tissues are associated with loss of immune privilege.^{B10}

The mediators of the immunosuppression in eyes are found in the aqueous humor, as first demonstrated by Kaiser et al., who suppressed various in vitro T cell assays with normal aqueous humor, and by Streilein and Cousins, who showed that when T cells primed for Th1 activity were pretreated with aqueous humor, they failed to mediate the expected inflammatory response in a local adoptive transfer of DTH assay in skin.^{A2,A3} More recently, it has been reported that primed T cells activated in the presence of aqueous humor were deflected from an expected Th1 response to a Th3 response. A8 Such aqueous humor-induced T cells produced TGF-β, suggesting a Th3 phenotype, and suppressed IFN-γ production by other, Th1 type cells. However, it was not shown, prior to the present work, whether these aqueous humor-induced regulatory T cells can suppress DTH.

Several factors in aqueous humor have the potential to influence effector T cell activities^{A1.} Of the many factors in aqueous humor, the present experiments examine the ability of two factors, alpha-melanocyte stimulating hormone (α-MSH) and transforming growth factor-β2 (TGF-β2), to induce regulatory T cells.^{A8-A11} In the presence of α-MSH, activated primed T cells proliferate but are suppressed in their IFN-γ production.^{A10} This effect of α-MSH, which is independent of IL-4, has suggested that α-MSH mediates differential responses in T cells to TCR-stimulation. Recently it has been found that TGF-β mediates its own production by T cells.^{A11} These past findings failed to show whether or not these immunosuppressive factors could have a role in the apparent induction of regulatory T cells by aqueous humor. The findings presented here definitively show that α-MSH alone, or α-MSH in conjunction with TGF-β2, mediate(s) induction of TGF-β-producing, regulatory T cells that suppress DTH and may be Th3 cells. In addition to the direct immunosuppressive effects of these aqueous humor factors, the regulatory T cells they induce may also contribute to the normal immunosuppressive microenvironment of the eye by the cells' TGF-β production and suppression of Th1 cell activity. The results here suggest that within the normal ocular microenvironment, there is a potential for Th3 cell induction that supports the immunosuppressive micro-environment of the eye and that possibly mediates peripheral tolerance to ocular autoantigens.

More specifically, α-MSH, which occurs in aqueous humor, has recently been found to suppress IFN-γ production by, but not proliferation by, activated effector T cells.^{B11} This suggests that α-MSH may regulate regional induction of specific effector T cell responses. The thirteen-amino acid long α-MSH (1.6 kDa MW) is encoded within the pro-opiomelanocortin hormone (POMC) gene, and is released from the POMC protein through two endoproteolytic cleavage steps.^{B12,B13} It has a fundamental role in modulating innate host defense mechanisms in mammals, which contrasts to its original description as an amphibian melanin-inducing factor. Systemic and central injections of α-MSH suppress innate inflammatory responses induced by endotoxin, IL-1 and TNF (as opposed to adaptive, T cell-mediated immunity). Thus, α-MSH suppresses macrophage-reactive oxygen intermediates and nitric oxide generation, as well as production of inflammatory cytokines.^{B16-B21} In addition, B-MSH induces its own production and receptor expression on the macrophages promoting autocrine suppression of inflammatory-macrophage activities. Also, α-MSH suppresses macrophage and neutrophil chemotactic responses to chemokines and microbial chemoattractants.^{B19, B22} Macrophages, keratinocytes, centrally derived neurons, and possibly any cell that can synthesize POMC, are sources of α-MSH.^{B21, B23, B24} Normal mammalian aqueous humor (the fluid filling the ocular anterior chamber) constitutively expresses, on average, 30 pg/ml of α-MSH.^{B7}

Much is known about various immunomodulatory effects of α-MSH and of aqueous humor, which contains not only α-MSH but also TGF-β2 and many other factors. However, prior to the present work, much remained unknown about the role of α-MSH and TGF-β2 in regulating T-cell mediated inflammation through T cell networks.

### BRIEF SUMMARY OF THE INVENTION

The present work shows that α-MSH mediates the induction of TGF-β-producing, CD4+/CD25+, regulatory T cells that suppress the activation of other, effector T cells. Thus, α-MSH suppresses T cell-mediated inflammation and mediates selective production of T cell lymphokines. TGF-β2 enhances α-MSH mediated induction of regulatory T cells while TGF-β1 suppresses that induction.

The present invention relates to the discovery that treatment of primed T cells (also called memory or armed T cells) with certain immunomodulating factors, either alpha-Melanocyte Stimulating Hormone (α-MSH) alone or in conjunction with Transforming Growth Factor-β2 (TGF-β2), activates regulatory T cells that express both the T helper marker, CD4, and the T cell activation marker, CD25, and produce TGF-β (suggesting a Th3 phenotype). These regulatory T cells suppress activation of other inflammation-mediating T cells (primarily of the Th1 type). Such α-MSH treatment has induced in vitro activation of regulatory T cells specific to a particular antigen, e.g., an ocular autoantigen. These α-MSH-induced regulatory T cells were injected into mice at the same time that they were being immunized to induce experimental autoimmune uveitis (EAU), or when the mice were about to have symptoms of EAU. In both cases, EAU in those mice was suppressed—the mice showed no symptoms of autoimmune disease.

Therefore, the present invention provides a treatment for any autoimmune disorder or disease. Either α-MSH alone or a combination of α-MSH and TGF-β2 may be used, under certain conditions, to generate regulatory T cells against any autoantigen implicated in an autoimmune disease. Induction of antigen-specific regulatory T cells by α-MSH may also be used to prevent transplant graft rejection. Since the regulatory T cells activated by α-MSH are antigen-specific, they can also be generated against transplant antigens that are targeted by the immune system during transplant rejection.

Regulation of T cell activity is needed for maintaining tolerance to autoantigens. One of the regulatory mechanisms is mediated by factors produced by cells within a localized tissue microenvironment. One of these regulating factors is the neuropeptide, α-melanocyte stimulating hormone (α-MSH), which suppresses immunogenic inflammation. Th1 cells are suppressed from mediating inflammation when they are activated in the presence of α-MSH. Although there is proliferation, α-MSH suppresses IFN-γ production and possibly the secretion of IL-4 by T cell receptor (TCR)-stimulated, primed T cells. Such α-MSH treated T cells produce enhanced levels of TGF-β2. These TGF-β2-producing T cells have Th3 cells characteristics and suppress IFN-γ production by other activated Th1 cells. Therefore, α-MSH suppression of Th1 cell activity is a result of 0-MSH deviating the Th1 response into a regulatory T response, i.e., a Th3 response. The presence of α-MSH enhanced tyrosine phosphorylation of CD3ζ chains, but not of CD3ε chains. Hence, α-MSH appears to mediate immune deviation through induction of differential TCR-associated signals in T cells. The ability of α-MSH to mediate induction of regulatory Th3 cells implies that this neuropeptide has an important systemic and regional role in mediating and maintaining peripheral tolerance, especially in such tissues as the eye and the brain, which contain constitutive levels of α-MSH.

Even though the regulatory T cells are activated by α-MSH in an antigen-specific manner, their action is non-specific and general to the site of their activation. That is, a regulatory T cell is activated by the presence of a specific antigen to which that T cell has been primed, and by the presence of α-MSH, but once activated, it releases factors that are immunosuppressive generally, i.e., factors that suppress the inflammatory activities of other, effector T cells, primarily Th1 cells.

Therefore, the immunosuppressive or immunoregulatory method of the invention does not require generating regulatory T cells to all the tissue antigens involved in the autoimmune disorder or the transplantation being regulated. The regulatory T cell induction procedure can be standardized to a specific antigen that is injected into the autoimmune-diseased site or transplant site. Any accessible tissue site that may suffer from damaging immune responses, can be treated according to the methods of the invention, without having to know the exact antigen triggering the immune response causing the disease or graft rejection. In many cases of autoimmune disease, there is no clear characterization of the targeted autoantigen.

Thus, the present invention also provides a kit for culturing and treating T cells (e.g., harvested from peripheral blood) with α-MSH in the presence of a specific antigen and antigen presenting cells. After incubation, the α-MSH-treated T cells can be collected and injected back into the patient. The kit comprises α-MSH, a specific, target antigen, and an article of manufacture comprising instructions for how to use the α-MSH and target antigen to generate regulatory T cells. Optionally, the kit may also include T cell culture media conducive to generation of the CD4+/CD25+, TGF-β-producing T cells of the invention. TGF-β2 can also be included in the kit. The target antigen can be an autoantigen, so as to generate a regulatory T cell that specifically recognizes the autoantigen and re-establishes tolerance to that antigen. Alternatively, the target can be a ‘surrogate’ antigen, which would still generate regulatory T cells of the invention that are effective for down-regulating an autoimmune response or a host-versus-graft response, in that the regulatory T cells would still produce TGF-β and other cytokines necessary to down-regulate a T cell-mediated inflammatory response. Preferably, at least two samples of the target antigen should be included in the kit, one sample for adding to the T cell cultures in which regulatory T cells are to be generated, and one sample for injection into the diseased tissue site of the patient or the transplantation site of the graft recipient.

The invention also encompasses a gene therapy protocol for treating autoimmune disease. Tissue cells are transfected with genetic material that gives them the ability to produce and secrete α-MSH. Depending on the method of cellular transfection, the ability of the cells to produce α-MSH can be made to be short-term and temporary, or long-term and permanent. Temporary α-MSH-producing ability would result from “episomal transfection”, whereas the long-term approach integrates the transfecting material into the cell's chromosome(s).

The episomal transfection approach is preferred, as it carries a very low, nearly improbable, risk of transformation of the transfected cells into cancer cells. The transfecting materials could be applied directly to the eye as a mixture of lipids and genetic material, and would enter into the ocular tissues and into the anterior chamber and retina. If the plasmid is properly constructed, the transfected cells become a source of α-MSH. In this way, the immunoregulatory activity of α-MSH can be established in a localized tissue microenvironment at a level that will: (1) down-regulate or suppress T cell-mediated inflammation, and (2) induce regulatory activity by primed T cells (e.g., Th3 cells) being activated at the tissue site. Turning on or increasing α-MSH production within an eye suffering from autoimmune uveitis, would suppress the inflammation of the uveitis and re-establish the eye's immune privilege. Also, the ability of episomally transfected cells to make α-MSH would taper off in time, as cells tend to discard episomal genetic matter. α-MSH-induced regulatory T cells show some evidence of being stable and relatively long-lived. Therefore, there would appear to be little need for continuous treatment (i.e., repeated episomal transfection with genetic material for expressing α-MSH). The frequency of such treatment would, however depend on the conditions that produced the autoimmune disease in the first place.

The invention also provides gene therapy involving α-MSH for use in transplantation. A graft is treated with the transfecting material prior to implantation. A graft transfected with and producing α-MSH may be used to mediate activation of regulatory T cells primed to transplantation antigens. Such an application of the present invention reduces and could eliminate the need for tissue-typing to determine graft donor and recipient compatibility. Graft transfection with α-MSH genetic material also permits the use of organs from any otherwise suitable donor, not only individuals having compatible major histocompatibility complex (MHC) antigens. In the transplantation setting, a more lasting treatment may be needed (i.e., chromosomal transfection with an α-MSH gene), since most transplanted tissues are not naturally immune-privileged like the eye.

In fact, in accordance with the methods of the present invention, it has unexpectedly been determined that α-MSH immunoregulation is through the melanocortin 5 receptor (MC5r) on primed T cells. Therefore, the present invention further provides a therapeutic treatment with x-MSH that exclusively targets the MC5r receptor for a more efficient and direct suppression of T cell-mediated inflammatory response with reduced side effects. Possible therapeutic approaches include using a synthetic analogue of C-MSH that targets the MC5r exclusively to manipulate T cell functionality while leaving other MC(1-4)ᵣ receptor dependent pathways and functions unmodified. An antibody or a fragment thereof that binds to MC5r receptor and delivers α-MSH, an analogue or an agonist thereof, to the bound MC5r receptor may be used in accordance with the present invention. Moreover, such synthetic analogues or α-MSH associated antibodies that target the MC5r receptor exclusively to block only α-MSH effects on effector T cells and not activate MC5r associated intracellular signalling pathways are also within the scope of the present invention. An ordinary skilled artisan may use standard techniques in the methods described herein below to generate such α-MSH associated synthetic analogues or antibodies and fragments thereof.

### BRIEF DESCRIPTION OF THE DRAWINGS

Other features and advantages of the invention will be apparent from the following description of the preferred embodiments thereof and from the claims, taken in conjunction with the accompanying drawings, in which:

FIG. 1 is a bar graph of primed T cell proliferation, as measured by H³-thymidine uptake (CPM), in response to T cell receptor stimulation (anti-CD3) in the presence of various concentrations of α-MSH (pg/ml);

FIG. 2 is a bar graph showing the extent of IFN-γ production by primed T cells TCR-stimulated in the presence of α-MSH;

FIG. 3 presents the results of flow cytometric analysis of intracellular IFN-γ and IL-4 production by primed T cells TCR-stimulated in the presence of α-MSH;

FIG. 4 is a bar graph of TGF-β production (pg/ml) by primed T cells TCR-stimulated in the presence of various concentrations of α-MSH (pg/ml);

FIG. 5 is a bar graph showing production of IFN-γ by primed T cells in response to TCR stimulation alone or in the presence of α-MSH;

FIG. 6 is a bar graph similar to FIG. 5, but shows the long-term effect of α-MSH treatment on the TCR-stimulated primed T cells (IFN-γ production in response to TCR re-stimulation on day 5 after α-MSH treatment);

FIGS. 7A and 7B show the results of SDS-PAGE analysis of primed T cell lysates after incubation under various conditions: with or without α-MSH and with or without TCR stimulation, followed by immunoblotting with anti-phosphotyrosine antibody;

FIG. 8 depicts a bar chart showing DTH response in mice as measured by ear swelling, i.e., change in ear thickness (μm), as a function of the type of T cells administered, including whether or not the mice were injected with activated OVA-primed T cells treated with aqueous humor (“Regulatory T cells; AqH/OVA”);

FIG. 9 is a graph plotting percent proliferation of T cells in response to varying concentrations of TGF-β1 or TGF-β2, with or without α-MSH;

FIG. 10 is a graph potting percent proliferation as a function of the time at which either TGF-β1 or TGF-β2 was added (Hours TGF-β Added), after TCR-stimulation in the presence of α-MSH;

FIG. 11 is a bar chart showing total TGF-β production in cells treated with α-MSH and either TGF-β1 or TGF-β2, as a function of the time at which the TGF-β was added;

FIG. 12 shows the percent suppression in IFN-γ in activated effector T cells. TGF-β and α-MSH induce regulatory T cell activity;

FIG. 13 depicts a bar chart showing DTH response in mice as measured by ear swelling, i.e., change in ear thickness (μm), as a function of the type(s) of T cells administered, including whether or not the mice were injected with activated, OVA-primed T cells treated with α-MSH and TGF-β2 (“Regulatory T cells; α-MSH/TGF-β2 OVA”);

FIG. 14 shows the mean uveitis score in α-MSH-treated and untreated mice afflicted with experimental autoimmune uveitis (EAU);

FIG. 15 shows the results of an ocular fundus examination of mice for α-MSH suppression of EAU in B10.RIII mice immunized with IRBPp-primed T cells;

FIGS. 16A to 16E show the results of a cell proliferation assay on the effects of α-MSH on TGF-β producing regulatory T cells, IFN-7, IL-4, and IL-10;

FIG. 17 shows the results of a sandwich ELISA for IFN-7 suppression by α-MSH treated T cells;

FIGS. 18A to 18C show that α-MSH affects primed T cells independent of TCR-activated phosphorylation through the melanocortin-5-receptor (MC5r). FIG. 18A shows CD3ζ and CD3ε levels activated with anti-TCR antibody 2C11 in the presence of absence of α-MSH. FIG. 18B shows the results of primed T cells activated in the presence of α-MSH probed with anti-MC5r antibody. FIG. 18C shows the results of a sandwich ELISA for IFN-γ with T cells enriched from primed lymph nodes activated with anti-TCR antibody 2C11 in the presence of α-MSH and anti-MC5r antibody; and

FIGS. 19A and 19B show α-MSH induction of CD4⁺ and CD25⁺ regulatory T cells. FIG. 19A shows the results of a two-color flow cytometry after staining with anti-CD4 and anti-CD25. FIG. 19B shows IFN-γ levels of T cells activated in the presence of α-MSH and stained and sorted based on the co-expression of CD4 and CD25.

### DETAILED DESCRIPTION OF THE INVENTION

Regulation of T cell activity is needed for maintaining tolerance to autoantigens. One of the regulatory mechanisms is mediated by factors produced by cells within a tissue microenvironment. One of these regulating factors is the neuropeptide, α-melanocyte stimulating hormone (α-MSH), which is shown by the work herein to suppress immunogenic inflammation. As demonstrated in Example I, Th1 cells are suppressed from mediating inflammation when they are activated in the presence of α-MSH. Although there is T cell proliferation, α-MSH suppresses IFN-γ production and possibly the secretion of IL-4 by T cell receptor (TCR)-stimulated, primed T cells. Such α-MSH treated T cells produce enhanced levels of TGF-β. These TGF-β-producing T cells are characteristic of Th3 cells, and suppress IFN-γ production by other, activated Th1 cells. Therefore, it is shown here that α-MSH suppression of Th1 cell activity results from α-MSH apparently deviating the Th1 response into a Th3-like response. α-MSH enhances tyrosine phosphorylation of CD3ζ chains but not of CD3E chains. This phenomenon suggests that α-MSH could mediate immune response deviation through induction of differential, TCR-associated signals in T cells. The ability of α-MSH to mediate induction of TGF-β-producing, regulatory T cells implies that this neuropeptide has an important systemic and regional role in mediating and maintaining peripheral tolerance, especially in tissues such as the eye and the brain where α-MSH is constitutively present.

The ocular microenvironment is an extreme example of regional immunity. Within its microenvironment, expression of delayed type hypersensitivity (DTH) is suppressed. This immunosuppression is mediated in part by the constitutive expression of α-MSH in aqeuous humor. α-MSH has been found to suppress the production of IFN-γ by activated effector T cells (Th1).

The experiments of Example II were undertaken to determine whether aqueous humor-induced regulatory T cells could function in vivo. These regulatory T cells were examined for their ability to suppress adoptive transfer of delayed-type hypersensitivity (DTH). In addition, two aqueous humor factors, α-MSH and TGF-β2, were examined for their respective ability to induce regulatory T cells.

Primed T cells were treated with aqueous humor, α-MSH, TGF-β1, or TGF-β2 in Example II. These treated T cells were assayed for regulatory activity by injecting them intravenously (i.v.) along with inflammatory Th1 cells into syngeneic mice. Antigen-pulsed, antigen presenting cells (APC) were injected into the pinna of the mouse ear and swelling was measured 24 hours later. Primed T cells were also activated in vitro in the presence of α-MSH, TGF-β1 or TGF-β2, and were assayed for proliferation and TGF-β production along with their ability to suppress DTH.

The Example II results show that aqueous humor-treated T cells suppressed DTH mediated by Th1 cells. Maximum regulatory T cell activity was induced when primed T cells were activated in vitro in the presence of α-MSH, followed approximately 4 hours later with addition of active TGF-β2. Such T cells proliferated and produced TGF-β, suggesting that α-MSH and TGF-β2 induced activation of Th3 cells. No regulatory T cell activity could be induced in the presence of TGF-β1 (alone or in the presence of α-MSH). Therefore, not only do α-MSH and TGF-β2 have direct immunosuppressive effects. Additionally, through the constitutive production of α-MSH and TGF-β2, the ocular microenvironment can mediate induction of regulatory T, possibly Th3, cells that can contribute further to the immunosuppressive microenvironment and immune privilege of the eye, through their production of TGF-β and by their ability to suppress activation of Th1 cells. Such a mechanism of immunosuppression may mediate the peripheral tolerance to ocular antigens that is needed to prevent induction of ocular autoimmune diseases.

In light of the finding, in Example I, that α-MSH can mediate induction of TGF-β-producing, regulatory T cells, the experiments in Example III were conducted to examine α-MSH's ability to suppress T cell-mediated inflammation (e.g., as in autoimmune disease) and to regulate lymphokine production by effector T cells. When α-MSH was injected intravenously (i.v.) into mice at the time of peak retinal inflammation, the severity of experimental autoimmune uveitis (EAU) was significantly suppressed. Effector T cells that were activated in vitro in the presence of α-MSH, proliferated and produced IL-4 as well as enhanced levels of TGF-β. However, their IFN-γ and IL-10 production was suppressed. The α-MSH-treated T cells functioned as regulatory T cells by suppressing in vitro IFN-γ production by other inflammatory T cells. This regulatory activity was the function of α-MSH-treated, CD4⁺ CD25⁺ T cells. Therefore, α-MSH mediates immunosuppression by inducing a differential expression of lymphokine production and by inducing activation of regulatory functions in T cells. This implies that α-MSH may take part in regional mechanisms of immunosuppression and possibly peripheral tolerance. Thus, α-MSH can be used to suppress autoimmune disease and possibly to re-establish tolerance to autoantigens.

The experimental results presented herein, demonstrate that a population of T cells expressing on their surface the cell surface proteins CD4 and CD25, expand when activated in the presence of α-MSH. Such CD4⁺ CD25⁺ T cells are known to be regulatory in activity. Cell surface staining and sorting of the α-MSH treated primed T cells show that the regulatory T cells are, indeed, CD25⁺ CD4⁺ T cells. In essence, c-MSH can mediate the induction of CD25+ CD4+ regulatory T cells. This observation suggests that a loss in peripheral blood CD4/CD25-positive cells, in response to presented autoantigen, could indicate a patient's susceptibility to a specific autoimmune disease. Therefore, the level of CD4/CD25-positive T cells in peripheral blood could serve as a prognostic indicator for an individual's susceptibility to autoimmune disease or relative risk of rejecting a transplant, and could also be used to asses the effectiveness of α-MSH treatment in that individual.

The following examples are presented to illustrate the advantages of the present invention and to assist one of ordinary skill in making and using the same. These examples are not intended in any way otherwise to limit the scope of the disclosure.

### EXAMPLES

### Exemplary Materials and Methods

Reagents and Animals. The experiments used synthetic α-MSH (Peninsula Laboratories, Belmont, Calif.); recombinant TGF-β2 and soluble TGF-β receptor type-two (R&D Systems, Minneapolis, Minn.); the following monoclonal antibodies: anti-CD4 (RM4-4), anti-CD25 (IL-2-receptor-α; 7D4) and anti-CD3ε (145-2C11) (Pharmingen, SanDiego, Calif.). B10.A and B10.RIII (Jackson Laboratories Bar harbor, ME) and BALB/c (institute breeding program) female mouse strains 4 to 8-weeks-old were treated with approval by the institutional animal care and use committee in accordance with the US Animal Welfare Act.

Antibodies and Cytokines. For TCR-stimulation, anti-CD3ε antibody 145-2C11 from Pharmingen (San Diego, Calif.) was used at a concentration that stimulated maximum proliferation and IFN-γ production by the primed Th1 cells (see below). In the sandwich ELISAs capture antibody and biotinylated-detection antibody pairs from Pharmingen were used for the IFN-γ, IL-4, and IL-10 assays. Recombinant mouse lymphokines used for standards in the sandwich ELISAs were from R&D Systems, Minneapolis, Minn. For flow cytometry Pharmingen PE-conjugated anti-CD4 antibody and FITC-conjugated anti-CD25 antibodies BVD4-1D11 (anti-IL-4) and XMG1.1 (anti-IFN-γ) were used. Synthesized α-MSH was from Peninsula Laboratories (Belmont, Calif.), and purified human TGF-β2 was from R&D systems. In addition, Santa Cruz Biotechnologies (Santa Cruz, Calif.) anti-CD3ε, anti-CD3ε_anti-melanocortin-3 receptor (MC3r), and anti-MC5r antibodies, plus rabbit anti-hamster IgG antibody (Sigma) and anti-phosphotyrosine antibody PY20 (ICN, Costa Mesa, Calif.) were used for the immunoprecipitation and immunoblotting assays.

TGF-β bioassay. To measure total TGF-β, 100 μl of conditioned media was pretreated according to standard procedures (24) with 10 μl 1N HCL to lower the media pH to 2 and incubated for 1 hour at 4° C. The acid was neutralized with 20 μl of a 1:1 mixture of 1N NaOH: 1M HEPES returning the culture supernatant to a pH 7.3. The transiently acidified conditioned media was then used in the Mv1Lu assay; diluted 1:4 in EMEM+0.5% FBS. To the wells of a Falcon 96-well flat-bottom plate, 100 μl of diluted transiently acidified samples were added with 100 μl of 1×10⁵ Mv1Lu cells (CCL-64; ATCC, Rockville, Md.). The plate was incubated for 20 hours at 37° C., 5% CO₂ followed by the addition of 20 μl of 50 μCi/ml 3H-thymidine and the plate was incubated for an additional 4 hours. Supernatant was discarded and 50 μl of 10× Trypsin-EDTA (Biowhittaker, Walkersville, Md.) solution was added to each well and the plate was incubated for 15 minute at 37° C. The cells were collected onto filter paper using a Tomtec Plate Harvester 96 and counts per minute (CPM) of incorporation 3H-thymidine was measured using a Wallac 1205 Betaplate Liquid Scintillation Counter. Cultures of known amounts of purified activated TGF-β1, 20 ng/ml to 0.2 pg/ml (R&:D Systems), were prepared in the same plate for calculating a standard curve to quantify the concentration of total TGF-β in the samples.

Flow Cytometry. For immunostaining and flow cytometry, T cells (2×10⁶ cells) were obtained from 24 hour cultures of enriched primed T cells TCR-stimulated in the presence of α-MSH as described above. The cells were centrifuged and washed once in 400 μl of brefeldin/PBS buffer (10 mM PBS, 10 μg/ml brefeldin A). The cells were resuspended in 100 μl of PBS/brefeldin and 100 μl of 4% paraformaldehyde/PBS fixing buffer. The cells were incubate at room temperature for 20 minutes with gentle agitation and washed once with 200 μl of brefeldin/PBS, centrifuged, and resuspended in 50 μl of PBS/saponin (10 mM PBS, 1% BSA, 0.1% Na Azide, 0.5% Saponin) and incubated 10 minutes at room temperature. To the cell suspensions 2 μg of FITC-conjugated anti-cytokine antibody (anti-IL-4 or -IFN-γ) or FITC-isotype control was added. The cells were incubated for 30 minutes at room temperature, washed twice with PBS/saponin, and washed once with PBS/BSA buffer (10 mM PBS, 3% BSA). The cells were resuspended in 50 ml of PBS/BSA buffer containing 2 μg of PE-conjugated anti-CD4 antibody and incubated for 30 minutes room temperature. The cells were centrifuged, resuspended in 1 ml of PBS/BSA buffer, and strained through nylon mesh into a snap cap tube with an additional 1 ml of PBS/BSA buffer washed through the mesh. The stained cells were analyzed by a Coulter Epics flow cytometer calibrated for two color fluorescence, and presented were the fluorescence of blast (proliferating) cells in two dimensions.

In another example, for immunostaining and fluorescence-activated cell sorting, T cells (2×10⁶ cells) from 24 hour cultures of the α-MSH-treated activated T cells were centrifuged and washed once in PBS/BSA buffer (10 mM PBS, 3% BSA). The cells were resuspended in 50 μl of PBS/BSA buffer containing 2 μg of PE-conjugated anti-CD4 and FITC-conjugated anti-CD25 antibodies and incubated for 30 minutes room temperature. The cells were centrifuged, resuspended in 1 ml of PBS/BSA buffer, and washed two times. The stained cells were sorted by a Coulter ELITE cell sorter calibrated for two color fluorescence. The cells were sorted into two populations, CD25⁺ CD4⁺ cells and the remaining cells (all CD4⁻ cells plus CD25⁻CD4⁺ cells). The sorted cells were used immediately in the in vitro regulatory T cell assay.

Assay For In Vitro Regulatory T Cell Activity. The α-MSH-treated TCR-stimulated T cells, as described above, were cultured for 48 hours. The plate was spun down at 250×g for 10 minutes and supernatant discarded. Freshly isolated, enriched in vivo primed Th1 cells (4×10⁶ cells/ml) mixed with 2C11 (1 μg/ml) were added (200 μl) to the wells of the α-MSH pre-treated, TCR-stimulated T cells and incubated for 48 hours. The culture supernatant was assayed for IFN-γ by sandwich ELISA. For long term cultures, primed T cells were TCR-stimulated in the presence of α-MSH for 48 hours. The plate was centrifuged and supernatant was exchanged for 200 μl of 2C11 (1 μg/ml) in fresh media, no x-MSH. The cultures were incubated for 72 hours and the conditioned media was again exchanged for fresh media and 2C11 antibody. The cultures were incubated for an additional 48 hours, centrifuged, supernatant discarded, and added were fresh in vivo primed Th1 cells and 2C11 antibody. Soluble TGF-β receptor II (sTGF-βRII, R&D Systems) was added to some of the cultures. These cultures were incubated for 48 hours and the culture supernatant was assayed for IFN-γ by sandwich ELISA.

Antigens. Ovalbumin (OVA; Sigma Chemical, St. Louis, Mo.); desiccated Mycobacterium tuberculosis (MT-Ag; Difco, Detroit, Mich.) were used to immunize the mice.

Aqueous Humor. Aqueous humor (AqH) was obtained from New Zealand White rabbits (Pine Acres Rabbitry, West Brattleboro, Vt.) with no observed ocular and systemic disease. Aqueous humor was passively drained from the ocular anterior chamber by paracentesis through a 27 gage perfusion set (Fisher Scientific, Pittsburgh, Pa.) that ended in a siliconized microcentrifuge tube (Fisher Scientific). Collected aqueous humor was used immediately in the assays.

T Cell Lines Specific for OVA. B10.A mice were immunized with 1 mg/ml OVA in complete Freund's adjuvant (Difco, Detroit, Mich.). After 7 days, popliteal lymph nodes were collected and T cells were isolated using a mouse CD3 enrichment column (R&D systems, Minneapolis, Minn.). T cells were cultured with irradiated (2000R) spleen cells (5×10⁶ cells/well) from syngeneic B10.A mice in the presence of OVA (300 μg/ml) for 7 days. The T cells were seeded at 2×10⁶ cells/well in a 24 well plate (Corning, Corning, N.Y.) with completed Dulbecco's minimal essential medium (Biowhitter, Walkerville, Md.) supplemented with 10% fetal bovine serum (Hyclone, Logan, Utah), 0.05 mM 2-mercaptoethanol (Gibco/BRL, Grand Island, N.Y.), 25 mM HEPES (Biowhitter), 50 μg/ml Gentamycin (Sigma Chemical), 5 μg/ml L-Asparagine (Gibco/BRL), 5 μg/ml L-Arginine (Gibco/BRL). The T cells were collected and restimulated with OVA and syngeneic irradiated spleen cells in the culture media containing 80 U/ml mouse recombinant IL-2 (R&D systems, Minneapolis, Minn.) and 4000 U/ml mouse recombinant IFN-γ (R&D systems). The T cells were restimulated with OVA once every 2 to 3 weeks in the presence of syngeneic spleen cells. Cultures of developing T cells were determined to be Th1 cells when the T cells produced only IFN-γ with no detectable IL-4 production when stimulated by OVA presenting APC in the absence of exogenous growth factors (IL-2, IFN-γ) and mediated inflammation in a standard adoptive transfer of DTH assay.

Sandwich Enzyme Linked Immunosorbent Assay (ELISA). The wells of a 96-well flexible microtiter plate (Falcon, Oxnard, Calif.) were coated with capturing monoclonal antibody (anti-IFN-γ; Pharmingen, San Diego, Calif.) to the cytokine being assayed. The plate was incubated overnight at 4° C. and was washed with a solution of phosphate buffer saline, 0.026 Tween-20 and 1% BSA (wash buffer) and blocked with PBS plus 1% BSA (PBS-BSA). The plate was incubated for 1 hour room temperature and washed. Samples were added to the wells and the plate was incubated for 3 hours at room temperature. The plate was washed 3 times and into each well 100 μl of 1.0 μg/ml of biotinylated-detecting monoclonal anti-IFN-γ antibody (Pharmingen) was added. The plate was incubated for 1 hour and washed 3 times. Strepavidin-β-galactosidase (Gibco/BRL), 100 μl, was added to the wells and the plate was incubated for 30 min and washed 5 times. The substrate chlorophenyl-red-β-D-galactoside (CPRG: Calbiochem, San Diego, Calif.) was added to the wells and color was allowed to develop for 30 min. The optical density of the converted CPRG was read on a standard ELISA plate reader at a wavelength of 570 nm. The INF-γ concentration of the standard samples were plotted against their OD to create a standard curve. Using this standard curve, the concentration of INF-γ in the assayed culture supernatant was determined from the OD of the test well and sample dilution factor.

Adoptive transfer and Delayed-type hypersensitivity (DTH). T cells from the draining lymph nodes of B10.A mice immunized 7 days previously with either OVA were isolated using a CD3 enrichment column (R&D Systems). The enriched primed T cells (4×10⁵ cells) were added to cultures containing aqueous humor (50% diluted in culture media) and antigen pulsed APC. The antigen pulsed APC were adherent spleen cells (1×10⁶ cells) from syngeneic naive mice pulsed overnight with antigen (OVA or MT-Ag) and washed with media before adding T cells and aqueous humor. The cultures were incubated for 24 hours at 37° C., 5% CO₂. In some experiments instead of aqueous humor, the T cells were added to cultures containing the antigen pulsed APC and 30 pg/ml α-MSH. After a 4 hour incubation, TGF-β (5 ng/ml) was added and the cultures were incubated for the remaining 20 hours. The cells were collected and assayed for regulatory activtiy in the adoptive transfer of delayed type hypersensitivity. The culture media was serum-free containing RPMI 1640, 1 mg/ml BSA, 1/500 dilution of ITS+ solution (Collaborative Biomedical Products, Bedford, Mass.).

T cells (2×10⁵ cells) from the inflammatory OVA T cell line cultures were injected along with aqueous humor or α-MSH and TGF-β treated T cells (2×10⁵ cells) into the tail veins of syngeneic (B10.A) mice in a volume of 200 μl. Within one hour antigen pulsed syngeneic APC (1×10⁵ cells) were injected into the right ear pinna of the mouse and ear swelling was measured with a micrometer (Mitsutoyo, Japan) at 24 and 48 hours. Maximum ear swelling occurred at 24 hours and these data are presented as the mean±SEM of the difference between ear thickness of the APC injected ear and the opposite ear injected with PBS alone, minus their respective original ear thickness. Significance was determined by Student's t test of p=0.05.

Primed T Cell In Vitro Assays. From draining lymph nodes of BALB/c mice immunized 7 days previously with MT-Ag, primed T cells were isolated using CD3 enrichment columns (R&D Systems). T-cells (4×10⁵ cells) suspended in serum-free media were added to the wells of a 96 well, round bottom plate (Corning). To the wells were added α-MSH (30 pg/ml) and anti-TCR antibody (2C11; 1 μg/ml) diluted in serum-free culture media. Various concentrations of TGF-β1 or TGF-β2 in media were added. In a second set of experiments TGF-β1 or TGF-β2 at a fixed concentration of 5 ng/ml were added to the wells at various times (0, 2, 4 and 6 hours) after addition of anti-TCR antibody. The cultures were incubated for 24 hours and 0.5 μCi of 3H-thymidine (NEM, Boston, Mass.) was added to the wells and the cultures were incubated for an additional 24 hours. The cells were collected and incorporated radiolabeled was measured by scintillation counting. Production of TGF-β by the treated primed T cells was done by centrifuging the culture plates 24 hours after the addition of anti-TCR antibody, removing the supernatant, washing the cultures once and adding fresh media. The cultures were incubated for an additional 24 hours and the culture supernatant was assayed for TGF-β using the standard CCL-64 bioassay for TGF-β activity as we have previously described.⁸

α-MSH treatment of primed T cells. BALB/c mice (institute breeding program) were immunized via a cutaneous foot injection with 0.5 mg desiccated Mycobacterium tuberculosis (Difco, Detroit, Mich.). All animal use in this report was approved by the Institutional Animal Care and Use Committee under the U.S. Animal Welfare Act of 1966 amended. From the draining popliteal lymph node, the T cells were enriched, 99% CD3⁺ by flow cytometry analysis, using a mouse T cell enrichment column (R&D Systems). Into the wells of a 96 well, round bottom plate (Corning, Corning, N.Y.) were added T cells (4×10⁵ cells), α-MSH (30 pg/ml) and 2C11 antibody (1 μg/ml) in serum-free culture media. Anti-melanocortin receptor 5 antibody (anti-MC5r) 1 μg/ml was added to some of the cultures. The cultures were incubated for 48 hours and the supernatants were assayed for lymphokines in using sandwich enzyme linked immunosorbent assays (ELISA) specific for IFN-γ, IL-4, IL-10, and using the standard CCL-64 bioassay for TGF-β. The serum-free culture media^{C23} was RPMI 1640, 0.1% BSA solution (Sigma Chemical, St. Louis, Mo.), and a 1/500 dilution of ITS+ solution (Collaborative Biomedical Products, Bedford, Mass.). For assaying proliferation, the T cell cultures were initially incubated for 24 hours and 20 μl of 50 μCi/ml of ³H-thymidine (NEM, Boston, Mass.) was added to the wells and the cultures were incubated for an additional 24 hours. The cells were collected onto filter paper using a Tomtec Plate Harvester 96, and radiolabel was measured using a Wallac 1205 Betaplate Liquid Scintillation Counter.

Experimental Autoimmune Uveitis and adoptive transfer of antigen-specific α-MSH treated T cells. Primed T cells were collected and enriched as described above; however, the primed T cells were from B10.RIII mice where they were immunized with 50 μg of interphotorecepter retinoid binding peptide 161-180 (IRBPp), or 100 μg of OVA emulsified with adjuvant. The enriched T cells (8×10⁵ cells/well) were cultured with antigen-pulsed APC with or without 30 pg/ml α-MSH in a flat bottom 96-well culture plate. The antigen-pulsed APC were naive adherent spleen cells (1×10⁶ cells/well) that were cultured with 5% FBS (Hyclone Laboratories, Logan, Utah) RPMI-1640 for 90 min in the 96-well flat bottom culture plate, washed twice with media, and incubated overnight with IRBPp 50 μg/ml, or OVA 100 μg/ml. Before using these cells as antigen-pulsed APC, they were washed twice with serum free media. The cultures of primed T cells and APC were incubated for 24 hours.

To induce EAU, the B10.RIII mice were immunized in the footpad, thigh, base of tail and the back with 50 μg of IRBPp emulsified with CFA containing 3.0 mg/ml of Mycobacterium tuberculosis H37RA^{C27}. On the same day of the immunization, mice were injected intravenously with 2×10⁵ T cells from the in vitro cultures. The retinitis was clinically assessed every 3 days starting 6 days after the immunization. The ocular fundus was examined by direct ophthalmoscopy following pupil dilation with 0.5% Tropicamide and Neo-Synephirine drops. The severity of inflammation was clinically graded on a 0 to 5 scale^{C28}. Retinas with no inflammation was scored 0, with only white focal lesions of vessels were scored 1, with linear lesions of the vessels within half of retina were scored as 2, with linear lesions of vessels over more than half of the retina were scored as 3, with severe chorioretinal exudates or hemorrhages in addition to the vasculitis were scored as 4, and retinas with subretinal hemorrhage or retinal detachments were scored as 5. No mouse under our housing and care ever reached a clinical score of 5.

Immunoprecipitation and Immunoblotting. Enriched primed T cells (2×10⁶ cells) in a 24 well Corning plate were TCR-stimulated with 2C11 in the presence of α-MSH (30 pg/ml) under serum-free conditions for 15 minutes. The T cells were collected, placed in a microcentrifuge tube and washed once with 10 mM Tris buffered saline (TBS) and lysed for 30 minutes in 100 μl of ice cold lysate buffer (10 mM TBS, 1% NP-40, 0.5% sodium deoxycholate, 0.1% SDS, 100 μg/ml phenylmethylsulfonyl fluoride (PMSF), 60 μg/ml Aprotinin, and 1 mM sodium orthovanadate). The cells were passed through a 21 gauge needles three times, and an additional 2 μl of 10 mg/ml PMSF was added. The tubes were incubated for an additional 30 minutes on ice and were centrifuged for 20 minutes, 4° C., 13,000×g. The supernatant was collected and used as total cellular lysate. The protein concentration was measured, and added to equal amounts of protein lysate were 20 μg/ml anti-CD3ζ antibody, or anti-CD3ε antibody plus rabbit anti-hamster IgG antibody and incubated overnight at 4° C. The rabbit anti-hamster IgG was needed for the CD3ε immunoprecipitation, since much of the CD3ε was bound by 2C11 a hamster anti-mouse CD3F and hamster antibody weakly binds protein-G. Protein-G sepharose beads (Pharmacia, Piscataway, N.J.) were added to the antibody containing lysates and incubated for 1 hour at room temperature with end over end agitation. The beads were centrifuged and washed 4 times with 10 mM TBS containing 0.5% sodium deoxycholate. After the final wash the beads were resuspended in 20 μl of distilled water and 20 μl non-reducing SDS Tris-glycine sample buffer (Novex, San Diego, Calif.), boiled for 5 minutes and applied into two wells (20 μl) of a 8-16% Tris-glycine polyacrylamide gel (Novex).

Following electrophoresis in a Novex Xcell II minicell and blot module, the proteins were transferred from the gel onto a nitrocellulose membrane (Novex) by electroblotting. The membrane was blocked with 1% BSA in 0.01 M TBS for 1 hour room temperature. The blocked membrane was placed in a sealable bag containing 5 ml of alkaline-phosphatase conjugated anti-phosphotyrosine antibody PY20 diluted 1/2000 in 1% BSA-TBS buffer. The membrane was incubated overnight at room temperature. The PY20 blotted membrane was washed 3 times with wash buffer containing 1% BSA-TBS and 0.05% Tween-20 and incubated with alkaline phosphatase substrate NBT-BCIP (Sigma Chemical) until bands appeared. The membrane was washed with distilled water. In parallel, some lanes on the membrane were immunoblotted with anti-CD3ζ antibody and alkaline phosphatase conjugated anti-mouse IgG (Sigma Chemical) to detect the relative mobility of CD3ζ protein. Membranes were digitally photographed and analyzed using NIH Image software to integrate the band intensities relative to band area minus background. To detect MC3r and MC5r expression by the T cells, primed T cells were lysed and anti-MC3r or anti-MC5r antibodies were used to immunoprecipitate and immunoblott their respective receptor proteins.

### Example I

α-MSH has No Effect on TCR-Stimulated T Cell Proliferation.

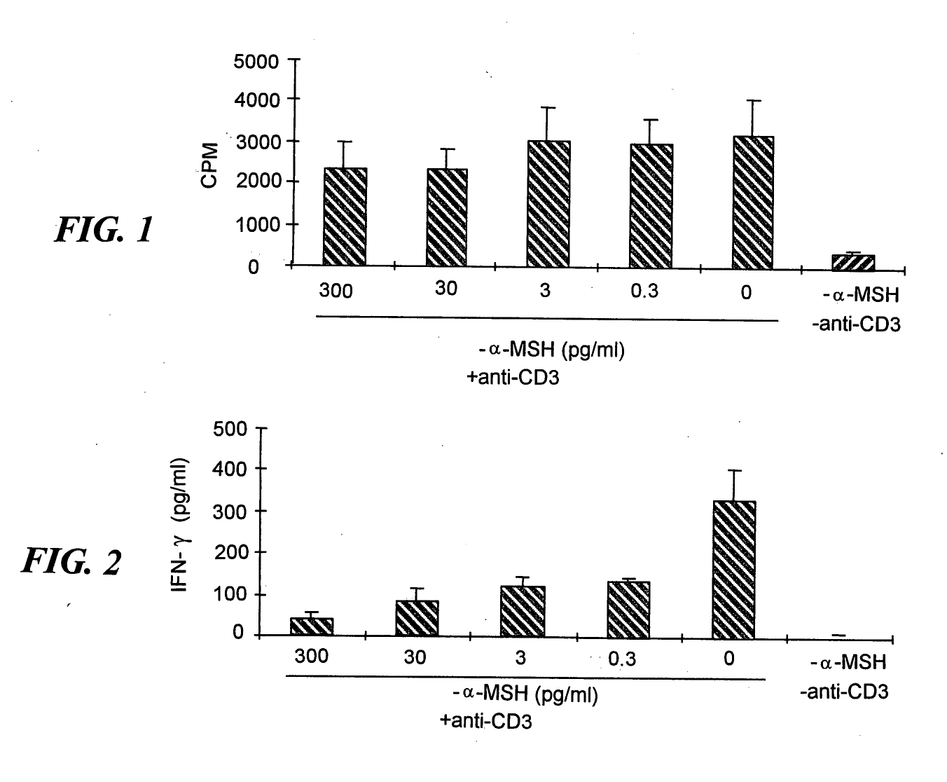

Since α-MSH was previously found to suppress IFN-γ production by antigen-stimulated Th1 cells⁹, we investigated whether α-MSH suppresses all TCR-associated activities or only IFN-γ production. Additionally, α-MSH has been shown to have the potential to affect both antigen presenting cells (APC) and T cells. This observation made it uncertain as to whether the T cells could be a direct target of α-MSH immunosuppressive activity. To eliminate the influence of α-MSH on APC activation of T cells, APC were removed by enriching for CD3⁺ T cells from lymph nodes primed to M. tuberculosis. Also, the T cells were stimulated with anti-CD3ε antibody, 2C11, at a concentration that stimulated the T cells to maximally proliferate and produce IFN-γ, a maximized in vitro Th1 cell response. To examine the possibility that our previously observed α-MSH suppression of INF-γ production was due to α-MSH suppression of Th1 cell activation, the enriched primed T cells were TCR-stimulated in the presence of α-MSH and proliferation was assayed. We found that α-MSH had no effect on TCR-stimulated Th1 cell proliferation (FIG. 1). Therefore, α-MSH has no effect on the TCR-associated signals mediating proliferation.

FIG. 1, depicts the proliferation of primed T cell (H³-thymidine uptake (CPM)), in response to T cell receptor stimulation (anti-CD3) in the presence of various concentrations of α-MSH (pg/ml). T cells from a draining lymph node of a BALB/c mouse immunized 7 days previously with M. tuberculosis were enriched and incubated (4×10⁵ cells) in serum-free media with anti-CD3ε (2C11) in the presence of declining concentrations of α-MSH for 24 hours. To the cultures was added 0.50 μCi of ³H-thymidine and they were incubated for an additional 24 hours. The cells were collected and counted by scintillation for incorporated radiolabel. The FIG. 1 results are presented as CPM±SEM of eight independent experiments.

α-MSH Suppresses IFN-γ Production and IL-4 Secretion by TCR-Stimulated Primed T cells.

Since TCR-stimulated proliferation was not affected by α-MSH, it is possible that α-MSH affects selective TCR-associated activities. We assayed the culture supernatant of the enriched primed Th1 cells TCR-stimulated in the presence of x-MSH for INF-γ and IL-4. IFN-γ production was suppressed when the primed T cells were TCR-stimulated in the presence of physiological concentrations of α-MSH, as shown in FIG. 2.

FIG. 2 is a bar graph showing the extent of IFN-γ production by primed T cells TCR-stimulated in the presence of α-MSH. Primed T cells were obtained, enriched and cultured as in FIG. 1 and incubated for 48 hours. The culture supernatants were assayed for IFN-γ by sandwich ELISA. The results are presented as pg/ml±SEM of eight independent experiments. In all assayed concentrations of α-MSH, levels of IFN-γ in the culture supernatant were significantly (p=0.05) suppressed in comparison to the levels of IFN-γ in the cultures of enriched primed T cells TCR-stimulated in the absence of α-MSH.

Also assayed was IL-4, since its production is considered an indication of Th2 cell activity countering Th1 IFN-γ production. We could not find any IL-4 in the supernatants of any TCR-stimulated T cell cultures (data not shown).

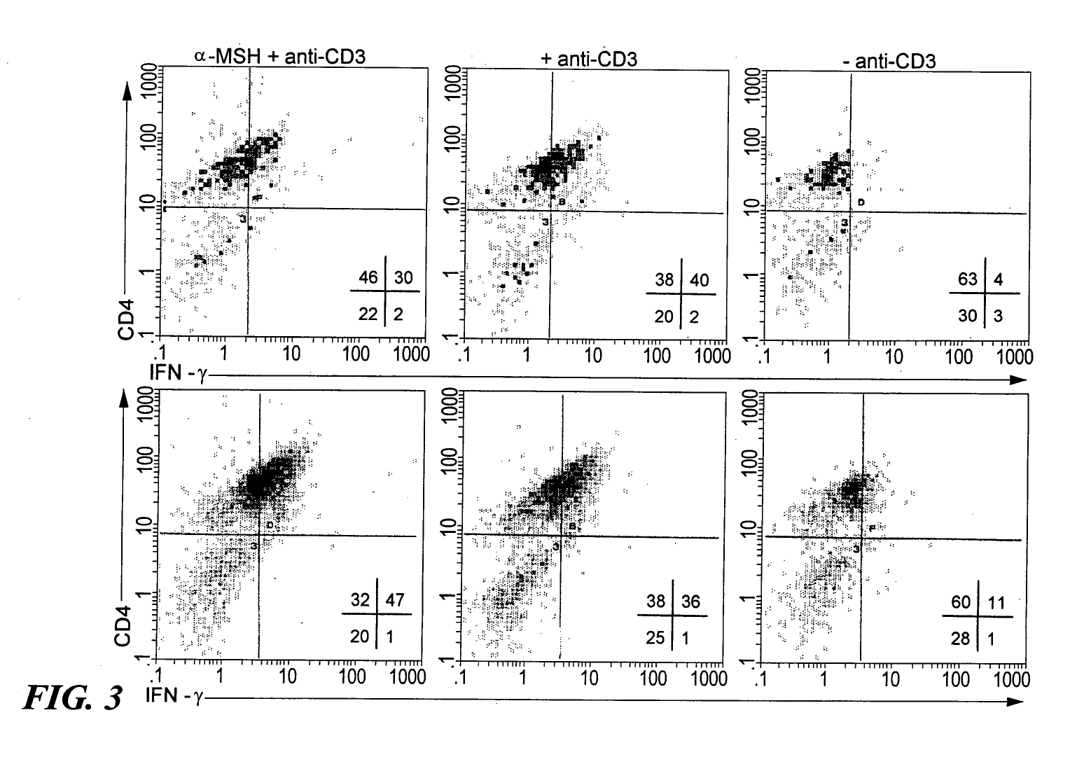

To further examine α-MSH suppression of IFN-γ production by T cells, the frequency of IFN-γ producing T cells activated in the presence of α-MSH was assayed by flow cytometry. The α-MSH treated TCR-stimulated T cells were stained for surface-expressed CD4 and for intracellular IFN-γ protein. The frequency of IFN-γ positive cells substantially shifted toward lower levels of intracellular IFN-γ staining in CD4+ T cells activated in the presence of α-MSH, as shown in FIG. 3. FIG. 3 presents the results of flow cytometric analysis of intracellular IFN-γ and IL-4 production by primed T cells TCR-stimulated in the presence of α-MSH. Primed T cells were obtained and enriched as in FIG. 1. The enriched T cells (2×10⁶ cells) were incubated with anti-CD3 in the presence of α-MSH (30 pg/ml) for 24 hours. Unstimulated, enriched T cells were cultured in media alone (-anti-CD3). The T cells were fixed with paraformaldehyde, permeabilized with saponin and stained for intracellular IFN-γ or IL-4 with PE-conjugated antibodies in saponin buffer. The surface of the cells was stained with FITC-conjugated anti-CD4. The stained cells were analyzed by two-color flow cytometry gating on the blastogenic, proliferating T cells. Quadrant lines were placed to separate CD4⁺ and CD4⁻ cells vertically and PE-conjugated isotype control on the horizontal. The cell density was equilibrated among the histograms. The histograms are all from one experiment representing similar results of four independent experiments. Percent of analyzed cell population is given for each quadrant in the lower right-hand of each histogram. The presence of IFN-γ stained T cells was limited to only the CD4⁺ T cells. Therefore, the decrease in IFN-γ detected in the culture supernatants was due to a decrease in the frequency of IFN-γ-synthesizing T cells stimulated in the presence of α-MSH.

Even though no IL-4 was detected in the culture supernatant by ELISA, there was a shift to higher levels of IL-4 staining in the CD4⁺ T cells stimulated in the presence of α-MSH (FIG. 3). Thus, the primed T cells activated in the presence of α-MSH expressed intracellular IL-4, but no IL-4 was detected in the culture supernatants. This observation suggests that α-MSH may promote induction of IL-4 protein synthesis, but that IL-4 secretion may be suppressed within at least the time span of these experiments (48 hours). Therefore, primed Th1 cells TCR-stimulated in the presence of α-MSH are suppressed in their IFN-γ production and are not fully deviated to a Th2 cell response.

α-MSH Enhances Production of TGF-β by Primed T Cells.

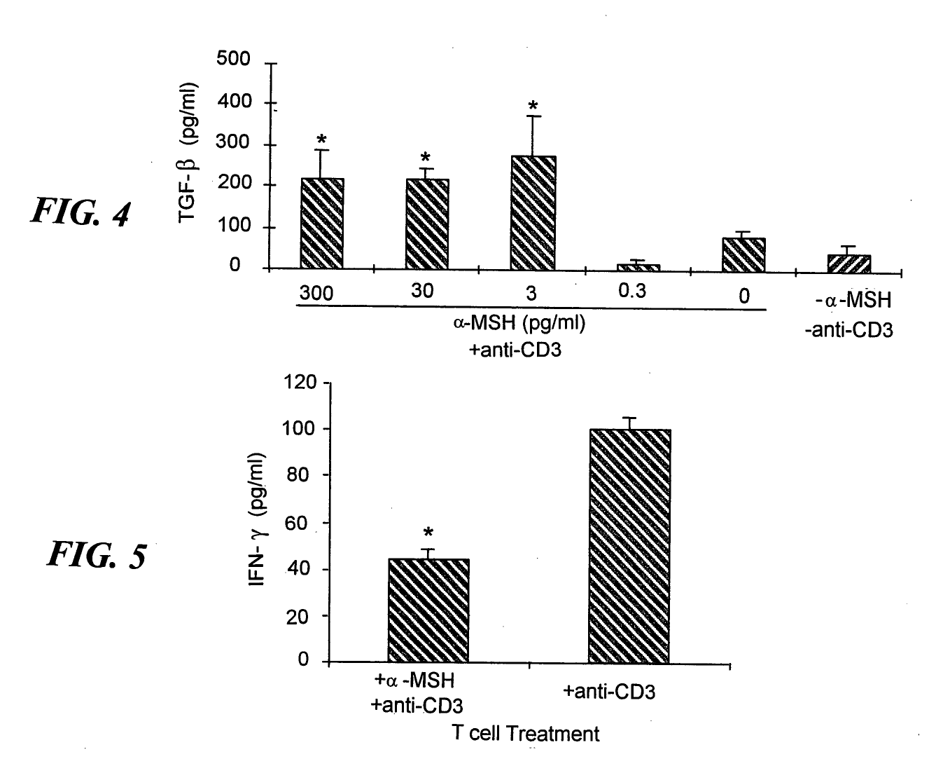

The oral tolerance models have suggested that TGF-β producing T cells have an important role in regulating and suppressing autoimmunity²⁵⁻²⁹. It has been suggested that they be considered a third type of T cells, to contrast their suppressive activity with the immune functions of Th1 and Th2 cells. Since the primed T cells TCR-stimulated in the presence of α-MSH did not produce or release the prototypical lymphokines for Th1 (IFN-γ) and Th2 (IL-4) cells, experiments were conducted to determine whether the α-MSH treated primed T cells could produce the Th3-associated lymphokine, TGF-β³⁰. The enriched primed T cells were TCR-stimulated in the presence of α-MSH as before, and the culture supernatants were assayed for total TGF-β using the TGF-β bioassay (FIG. 4).

FIG. 4 is a bar graph of TGF-β production by primed T cells TCR-stimulated in the presence of α-MSH. Primed T cells were obtained, enriched and cultured as in FIG. 1 under serum free conditions. After 48 hours of incubation the culture supernatant was collected and all TGF-β in the supernatant was activated by transiently acidified with 1N HCl neutralized by a 1:1 mixture of 1M HEPES: 1M NaOH. The transiently acidified supernatant was assayed for TGF-β using the standard mink lung endothelial cell bioassay. The results represented as TGF-β pg/ml±SEM of eight independent experiments. Significance (p=0.05) was determined by Student's t-test between TGF-β concentrations in cultures of activated α-MSH treated T cells and T cells activated in the absence of α-MSH.

Thus, TGF-β production was significantly enhanced in cultures of α-MSH-treated, TCR-stimulated primed T cells. Therefore, when in vivo, primed Th1 cells that are TCR-stimulated in the presence of α-MSH produce TGF-β, possibly IL-4, but not the expected IFN-γ. Such T cells are generally considered to be Th3 cells.

α-MSH Mediates Induction Of Regulatory T Cells.

If α-MSH is mediating induction of Th3 cell function, then these α-MSH treated T cells should be regulatory in activity. The T cells were TCR-stimulated in the presence of α-MSH as before, incubated, and then mixed with fresh TCR-stimulated primed Th1 cells. The amount of IFN-γ produced in cultures of activated primed Th1 cells was suppressed by 60% percent when the α-MSH treated T cells were mixed into the cultures, as shown in FIG. 5.

FIG. 5 shows the effect on primed T cell production of IFN-γ, of TCR stimulation alone or in the presence of α-MSH. α-MSH induces regulatory activity in TCR-stimulated primed T cells. Primed T cells (4×10⁵ cells) were enriched and activated with anti-CD3 in the presence (α-MSH+anti-CD3) or absence (+anti-CD3) of α-MSH (30 pg/ml). After 48 hours of incubation the cells were mixed with freshly enriched primed Th1 cells (4×10⁵ cells) and anti-CD3 antibody and cultured for 48 hours. The culture supernatant was assayed for IFN-γ by sandwich ELISA. Results are presented as IFN-γ pg/ml±SEM of eight independent experiments. Concentration of IFN-γ in cultures containing T cells activated in the presence of α-MSH was significantly suppressed (p=0.05) in comparison to cultures where none of the T cells had seen α-MSH.

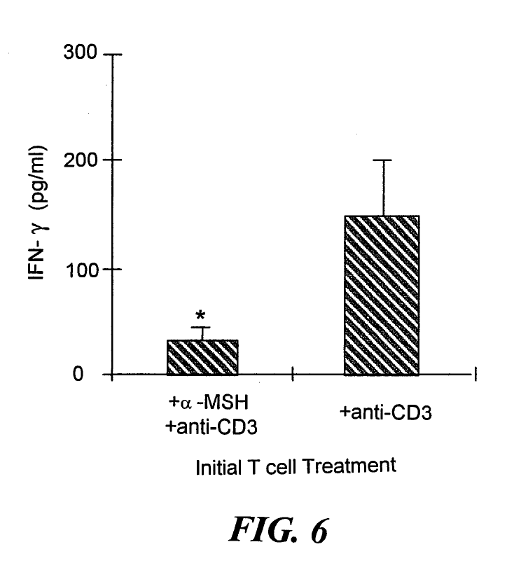

In addition, T cells initial TCR-stimulated in the presence of α-MSH and subsequently restimulated twice (Day 2 and Day 5) with anti-TCR antibody in the absence of α-MSH, still suppressed IFN-γ production by freshly activated primed Th1 cells (FIG. 6). FIG. 6 shows that α-MSH induces long term regulatory activity in TCR-stimulated primed T cells. Primed T cells (4×10⁵ cells) were enriched and activated with anti-CD3 antibody in the presence of 30 pg/ml α-MSH (α-MSH+anti-CD3) or absence of α-MSH (+anti-CD3). After 48 hours of incubation the cells were centrifuged and resuspended in fresh media with anti-CD3 antibodies and incubated for 72 hours (day 5). The cells were again washed and re-stimulated with anti-CD3 and incubated for an additional 48 hours (day 7). After the final incubation the treated T cells (4×10⁵ cells) were mixed with freshly enriched primed Th1 cells (4×10⁵ cells) and anti-CD3 antibody and cultured for 48 hours. The culture supernatant was assayed for IFN-γ by sandwich ELISA. Results are presented as IFN-γ pg/ml±SEM of eight independent experiments. Concentration of IFN-γ in cultures containing T cells initially activated in the presence of α-MSH was significantly (p=0.05) suppressed in comparison to cultures where none of the T cells had seen α-MSH. This observation suggests that α-MSH induces a permanent and stable regulatory function in the T cells. Therefore, α-MSH mediates induction of functionally active, regulatory Th3 cells.

α-MSH Enhances CD3ζ Chain Phosphorylation in Activated Primed T-Cells.

Since the induction of lymphokine production is linked to TCR-stimulation, there is a possibility that intracellular signals, emanating from the α-MSH-engaged melanocortin receptor, influence the tyrosine phosphorylation of CD3 molecules of the TCR. Such an influence could induce differential TCR-dependent responses^{31,32}. To demonstrate that α-MSH affects T cell response to TCR-stimulation, lysates of primed T cells TCR-stimulated in the presence of α-MSH were immunoprecipitated with either antibodies against CD3ζ or CD3ε chains, electrophoresed, and immunoblotted for phosphotyrosine.

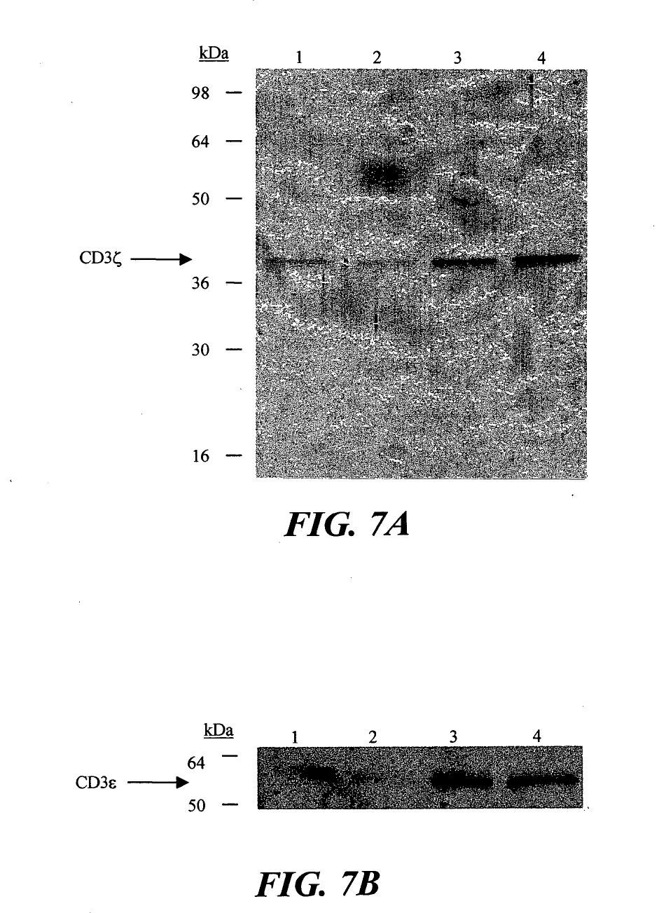

The results are shown in FIGS. 7A-B, and demonstrate the tyrosine phosphorylation of CD3ε and CD3ζ in primed T cells TCR-stimulated in the presence of α-MSH. The enriched primed T cells (2×10⁶ cells) were incubated with anti-CD3 in the presence of α-MSH (30 pg/ml) for 15 minutes. Unstimulated enriched T cells were cultured in media alone. The cells were washed and lysed. The lysates were immunoprecipitated with either anti-CD3ε antibodies (A) or anti-CD3ε antibodies (B). The immunoprecipitates were analyzed by non-reducing SDS-PAGE (on a 8-16% gradient gel), followed by transfer to a nitrocellulose filter and immunoblotting with anti-phosphotyrosine antibody: (1) Unstimulated primed T cells treated with α-MSH; (2) Unstimulated primed T cells incubated in media alone; (3) Enriched primed T cells TCR-stimulated in the absence of α-MSH; (4) Enriched primed T cells TCR-stimulated in the presence of α-MSH. The CD3ζ dimers were detected at 42 kDa (CD3ζ-ζ) and the CD3ε heterodimers were detected at 55 kDa (CD3ε heterodimers) by a simultaneous immunoblot of unstimulated T cell lysates run on the same gel using the anti-CD3ε or anti-CD3ε antibodies in the immunoprecipitation and immunoblot procedures. The relative intensities of the bands minus background, in lane order, for CD3ζ are 4, 1, 10, 67; and for CD3ε are 1, 1, 10, 10. The immunoblots are representative of three independent experiments.

The primed T cells, TCR-stimulated in the presence of α-MSH (Th3), had a 6.7-fold increase in CD3ζ tyrosine phosphorylation than the primed T cells stimulated in the absence of α-MSH (Th1 cells, FIG. 7A). In addition, c-MSH stimulated tyrosine phosphorylation of CD3ζ dimers by 4 fold in unstimulated primed T cells (FIG. 7A). The presence of α-MSH had no effect on the level of tyrosine phosphorylation of CD3ε chains of TCR-stimulated and unstimulated primed T cells (FIG. 7B). Therefore, it is possible that the induction of Th3 cells by α-MSH is due to α-MSH mediating a TCR-associated signal that is independent of TCR engagement. The results also further indicate that in vivo primed Th1 cells are receptive to α-MSH, resulting in their deviation into Th3 cells.

While the neuropeptide α-MSH suppresses Th1 cell responses, it induces regulatory activity in the same activated primed T cell population. Primed Th1 cells activated in the presence of α-MSH are suppressed in their expected production of IFN-γ and now produce TGF-β and possibly IL-4 indicating that the α-MSH has mediated a deviation of the activated Th1 cells into Th3 cells. Such T cells continue to proliferate and now function as immunoregulating T cells. It appears that α-MSH mediates some of its regulatory activity in T cells through differential tyrosine phosphorylation signals emanating from engaged T cell receptor proteins. The results here imply an important physiological role for α-MSH in regulating peripheral T cell activity. This role of α-MSH is important especially in tissues such as the brain and the eye where α-MSH is constitutively present.

The results show that α-MSH induces specific lymphokine production by the activated primed Th cells. Moreover, the results demonstrate that α-MSH can influence the functional developmental of primed Th cells. Such an observation falls more in line with the understanding that the functional differentiation of Th cells is mediated by their activation in the presence of specific cytokines, such as IL-12 for Th1 and IL-4 for Th2 development²⁹. Previously, α-MSH has been shown to act in a manner similar to IL-4, by suppressing induction of IFN-γ by TCR-stimulated Th1 cells⁹. However, the present results indicate that the influence of α-MSH on Th cell functionality is not an IL-4-like mediated deviation to Th2, but an induction of a Th3 response. It has recently been found that induction of Th3 cells can be mediated by IL-4 along with the neutralization or absence of IL-12³³. Therefore, the induction of a Th3 response could be mediated by α-MSH, acting on the Th cells as an IL-4-like agonist and as an IL-12 antagonist.

The potential for α-MSH to signal in lymphocytes in a manner similar to interleukins and other cytokines, has recently been found in B cells. There, α-MSH, through its melanocortin receptor (MC)-5, a G-protein associated receptor, activates the JAK1/STAT1 and STAT2 signal pathways³⁴. In this manner, α-MSH may act on T cells like other cytokines (i.e. IL-4) in regulating T cell development and differentiation into Th3 cells. For intracellular signaling, the present results indicate that the α-MSH receptor interacts with the TCR, leading to enhanced tyrosine phosphorylation of CD3ζ chains. The threshold of T cell activation is in relationship to the extent of phosphorylation of immune receptor, tyrosine-based, activation motifs (ITAM) on the TCR-CD3 proteins^{31,36}. The extent by which the ITAMs are phosphorylated influences initiation of the differential signaling pathways emanating from TCR engagement^{32,36}. The enhanced levels of CD3ζ chain tyrosine phosphorylation suggests that part of the effects of α-MSH on T cells, is through a signaling pathway of the TCR. It is possible that induction of regulatory Th cell activity by α-MSH is mediated through enhanced CD3ζ chain phosphorylation along with an α-MSH cytokine-like signal in the activated primed Th cells.

The result of activating in vivo primed Th1 cells in the presence of α-MSH is the induction of functional regulatory T cells. These T cells have been deviated by α-MSH away from their preset Th1 response (IFN-γ production). This deviation mediated by α-MSH may be an important function of α-MSH in tissues where there is a constitutive presence of α-MSH such as the eye and brain. The presence of bioactive α-MSH would promote the suppression of Th1 cells, including autoreactive T cells. Such α-MSH-mediated immunosuppression has been found in the immune privileged microenvironment of the eye^{3,23}. Whether α-MSH is an important factor in modulating T cell activity in other immune privileged tissues is to be seen; however, there is evidence that α-MSH may be an important regulator of T cell functions in the brain³⁷.

The most dramatic characteristic of α-MSH treated, primed T cells is their production of TGF-β. The activation of such T cells would elevate TGF-β concentration within a localized tissue microenvironment. The elevated TGF-β concentration could influence the course of immune, inflammatory and wound-healing responses³⁸⁻⁴⁴. The level of α-MSH activity in a tissue site could directly and indirectly, through TGF-β-producing T cells, regulate the induction, intensity, duration and resolution of an immune-mediated inflammatory response^{38,40}. Therefore, by elevating the concentration of α-MSH in a tissue site enduring a DTH response, α-MSH would suppress the inflammation in part by repressing IFN-γ production by Th1 cells, and by inducing TGF-β production by Th3 cells.

α-MSH is does not merely suppress Th1 cell activity and inflammation. It also is potentially a mediator of T cell differentiation into Th3 cells³⁰. Also, like Th3 cells, the α-MSH-treated T cells suppress the inflammatory activity of other activated Th1 cells. Therefore, if such regulatory T cells are generated to a specific antigen there is the potential to induce antigen-specific tolerance. Since the α-MSH-rich fluid of the eye can induce induction of Th3 cells⁴⁵, it is possible that α-MSH mediates tolerance to ocular autoantigens through the induction of Th3 cells within the ocular microenvironment. It has already been demonstrated that a systemic elevation of α-MSH, through an i.v. injection, at the time of immunization, can induce antigen-specific tolerance²¹. Therefore, since in the presence of α-MSH, activation of Th1 cells steers their development into CD4+/CD25+, Tgf-β-producing T cells (i.e., Th3 cells) that suppress the inflammatory activity of other activated Th1 cells, antigen-specific immunosuppression observed in the presence of α-MSH could very well be perceived as tolerance.

The immunosuppressive activity of α-MSH described here, suggests that if a tissue can be induced to secrete α-MSH, there would not only be prevention of an inflammatory response, but also the potential to induce immune tolerance to antigens within the tissue, through induction of antigen-specific the cells. Therefore localized α-MSH treatment¹² into tissue and organ grafts may induce tolerance to the transplanted tissue antigens. It is also possible that if α-MSH is delivered into sites of autoimmune disease, there would be, along with suppression of inflammation, restoration of tolerance to the autoantigens through α-MSH-induced autoantigen-specific Th3 cells. This evolutionarily conserved neuropeptide, α-MSH, demonstrates a connection between the nervous and immune systems that can be exploited therapeutically to regulate antigen-specific immune responses.

### Example II

Aqueous Humor Treated Primed T Cells Suppress DTH.

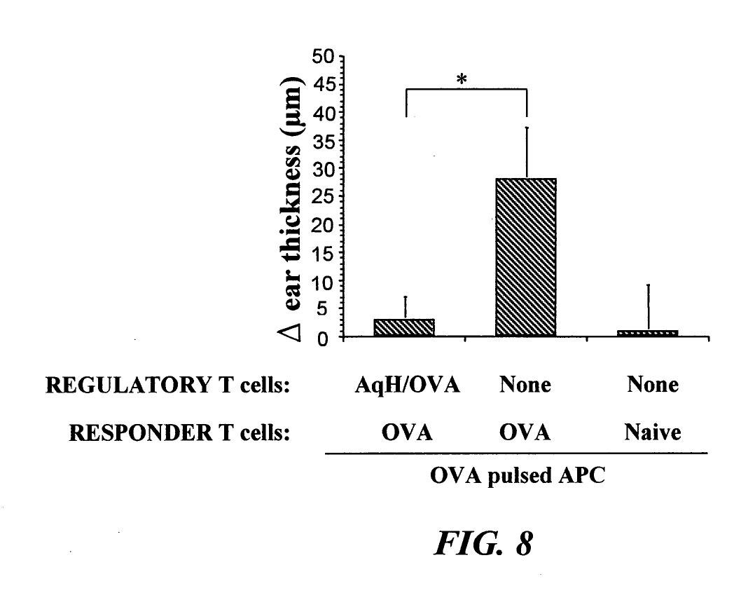

Previously we have demonstrated that primed T cells activated in the presence of aqueous humor, suppress in vitro IFN-γ produced by other Th1 cells⁸. This suggested that these aqueous humor-treated primed T cells should also suppress in vitro induction of DTH mediated by Th1 cells. To examine this possibility, aqueous humor-treated T cells, primed to OVA, were injected i.v. along with OVA-reactive Th1 cells. The aqueous humor-treated T cells significantly suppressed the inflammation mediated by the Th1 cells to OVA-pulsed APC that were injected into the pinna of the mouse ear (FIG. 8). Therefore, the regulatory T cells induced by aqueous humor suppressed the in vivo induction of DTH by other Th1 cells.

FIG. 8 is a bar chart showing DTH response in mice as measured by ear swelling, i.e., change in ear thickness (μm), as a function of the type of T cells administered, including whether or not the mice were injected with activated OVA-primed T cells treated with aqueous humor (“Regulatory T cells; AqH/OVA”). Aqueous humor-treated T cells suppress DTH mediated by other Th1 cells. Activated OVA-primed T cells treated with aqueous humor (Regulatory T cells; AqH/OVA) were injected i.v. with DTH mediating T cells (Responder T cells; OVA). OVA-pulsed APC were injected into the ear pinna and ear swelling was measured 24 hours later. The data represent two experiments with similar results and are presented as the percent difference (see methods) in ear thickness±SEM (n=5) (P=0.05).

Factors of Aqueous Humor Regulate TCR-Stimulated Proliferation of Primed T Cells.

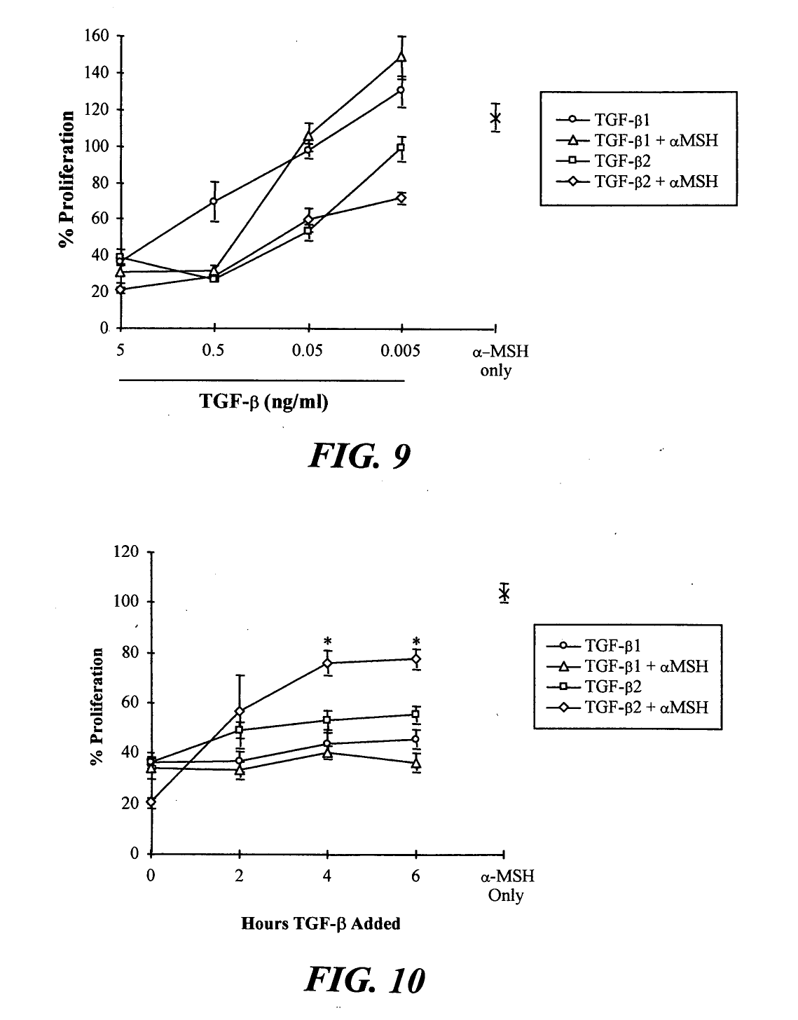

Aqueous humor contains constitutive levels of TGF-β2 and α-MSH 7912. To examine the effects of TGF-β2 in the presence of α-MSH on TCR-stimulated T cell proliferation, primed T cells were TCR-stimulated in the presence of α-MSH and active TGF-β1 or TGF-β2. Regardless of the presence of α-MSH, increasing concentrations of either TGF-β1 and TGF-β2 suppressed T cell proliferation (FIG. 9). It is interesting to find that low concentrations TGF-β1 had either no effect or enhanced T cell proliferation (FIG. 9).

FIG. 9 is a graph plotting percent proliferation of T cells in response to varying concentrations of TGF-β1 or TGF-β2, with or without α-MSH. There are concentration-dependent effects of TGF-β1 and TGF-β2 on in vitro T cell proliferation in the presence of α-MSH. Primed T cells were TCR-stimulated in the presence or absence of 30 pg/ml of α-MSH with either TGF-β1 or TGF-β2 (0.005-5.0 ng/ml). Proliferation was measured as counts per minute (CPM) of incorporated ³H-thymidine approximately 48 hours after TCR-stimulation. Data are presented as percent proliferation±SEM of eight independent experiments. Percent CPM was calculated as the CPM of sample divided by the CPM of untreated TCR-stimulated primed T cells (100% proliferation), minus background.

Since only active TGF-β2 can be added to the cultures, it is possible that the proliferative activity observed when the T cells are activated in the presence of whole aqueous humor, occurs because the T cells are influenced in time by increasing levels of latent TGF-β2 being activated in the cultures. This can be simulated by adding active TGF-β2 at various times after TCR-stimulation in the presence of α-MSH. Primed T cells were TCR-stimulated in the presence of α-MSH and, at various times afterwards with active TGF-β1 or TGF-β2. Here not only was it important whether α-MSH was present, but there was also a difference between the effects of TGF-β1 and TGF-β2, as seen in FIG. 10.

FIG. 10 plots the percentage proliferation as a function of the time at which either TGF-β1 or TGF-β2 was added (Hours TGF-β Added), after TCR-stimulation in the presence of α-MSH. There is a time-dependent effect of TGF-β1 and TGF-β2 on in vitro T cell proliferation in the presence of α-MSH. Primed T cells were TCR-stimulated in the presence or absence of 30 pg/ml α-MSH with TGF-β1 or TGF-β2 (5.0 ng/ml) added at different times after TCR-stimulation. Proliferation was measured as counts per minute (CPM) of incorporated 3H-thymidine 48 hours after TCR-stimulation. Data is presented as percent proliferation±SEM of eight independent experiments as explained in FIG. 2. MSH and TGF-β2 treated T cells significantly differed (p=0.05) from TGF-β2 only treated T cells.

Both TGF-β1 and TGF-β2 added from the start of TCR-stimulation through 6 hours later, suppressed T cell proliferation. However, if α-MSH was present, proliferation was recovered only with the addition of TGF-β2 at or later than 4 hours after TCR-stimulation (FIG. 10). Therefore, it is possible that in aqueous humor, the presence of α-MSH antagonizes the anti-proliferative activity mediated by activated TGF-β2.

TGF-β2 Enhances TGF-β Production by Primed T Cells Activated in the Presence of α-MSH.

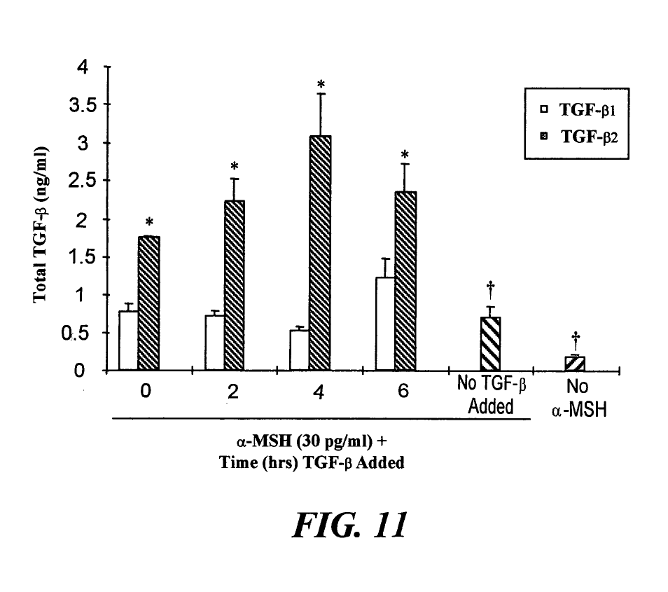

Another characteristic of primed T cells activated in the presence of aqueous humor is that they produce TGF-β.^{A8} FIG. 11 shows total TGF-β production in cells treated with α-MSH and either TGF-β1 or TGF-β2, as a function of the time at which the TGF-β was added. There is a time-dependent effect of TGF-β1 and TGF-β2 on TGF-β production by T cells activated in the presence of α-MSH. Primed T cells were TCR-stimulated in the presence of 30 pg/ml α-MSH, with 5.0 ng/ml of TGF-β1 or TGF-β2 added at different times after TCR-stimulation. Culture supernatants were assayed for total TGF-β levels, 48 hours after TCR-stimulation. Data are presented as TGF-β (ng/ml) SEM, from eight independent experiments.

TGF-β production was significantly different in primed T cell cultures, where TGF-β1 or TGF-β2 was added, from cultures where no TGF-β of any type was added (p=0.05).

The primed T cells TCR-stimulated in the presence of α-MSH produced enhanced levels of TGF-β (FIG. 11). Addition of TGF-β1 at various times after TCR-stimulation did not change the level of α-MSH-induced TGF-β production (FIG. 11). However, the addition of TGF-β2 at various times after TCR-stimulation did enhance α-MSH-induced TGF-β production by the primed T cells (FIG. 11). Therefore, the aqueous humor factors α-MSH and TGF-β2 mediate induction of TGF-β-producing T cells, which are potential regulatory T cells.

The Aqueous Humor Factors α-MSH and TGF-β2 Mediate Induction of Regulatory T Cells.

Since TGF-β2, when added about 4 hours after TCR-stimulation in the presence of α-MSH, can enhance TGF-βproduction by the treated T cells, it is possible that α-MSH and TGF-β2 induce activation of regulatory T cells. If regulatory T cells are activated, they should suppress IFN-γ production by other Th1 cells.

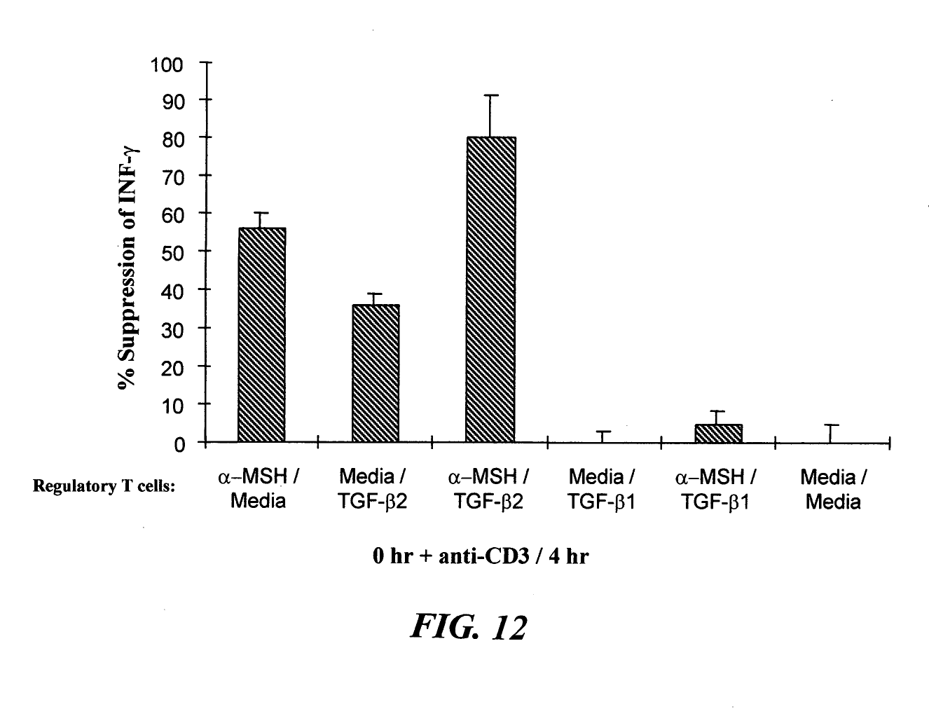

FIG. 12 shows the percent suppression in IFN-γ production in activated effector T cells. TGF-β and α-MSH induce regulatory T cell activity. Primed T cells were TCR-stimulated in the presence or absence of 30 pg/ml α-MSH with TGF-β1 or TGF-β2 (5.0 ng/ml) added 4 hours after TCR-stimulation. The treated T cells (Regulatory T cells) were added to cultures of freshly activated primed T cells. Culture supernatants were assayed for IFN-γ 48 hours after addition of treated T cells. Data are presented as percent suppression±SEM of IFN-γ produced by freshly activated T cells with no added regulatory cells, from eight independent experiments.

Primed T cells TCR-stimulated in the presence of α-MSH and then with TGF-β2 4 hours later, suppressed IFN-γ production by other Th1 cells when the treated primed T cells and Th1 cells were mixed into the same culture (FIG. 12). Individually, α-MSH, more than TGF-β2, induced regulatory T cell activity, but the addition of TGF-β2 enhanced the regulatory activity (FIG. 12). In contrast, TGF-β1 could not alone or with α-MSH induce regulatory T cells. Moreover, it appears that TGF-β1 antagonized α-MSH-mediated induction of regulatory T cells (FIG. 12). The induction of regulatory T cells by α-MSH with TGF-β2 corresponds with the recovered proliferation and enhanced levels of TGF-β production by the treated T cells, as seen in FIGS. 10 and 11.

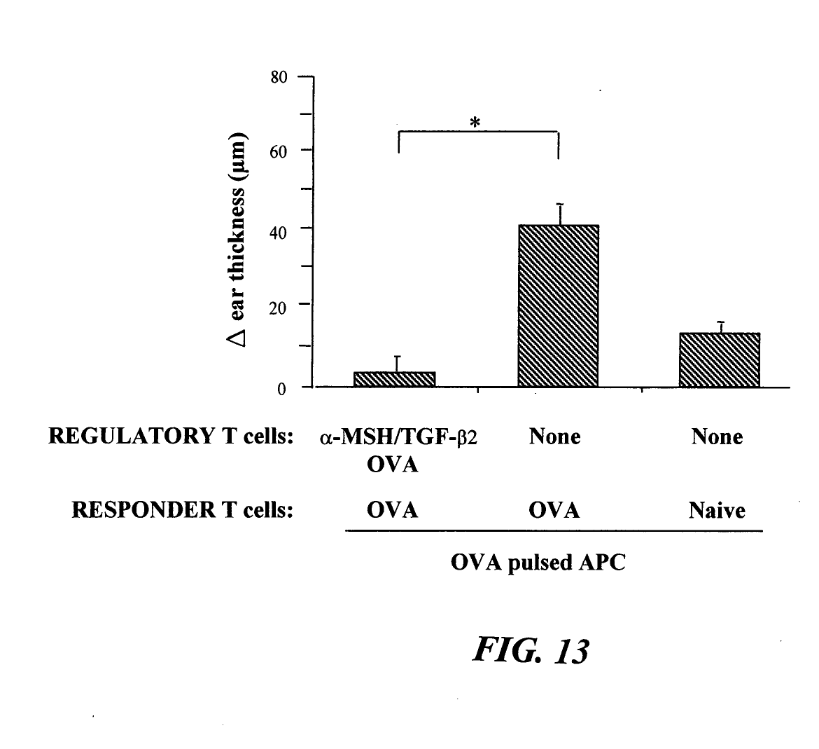

To demonstrate that these factor-induced regulatory T cells could, like aqueous humor-induced regulatory T cells, suppress DTH, primed T cells treated with α-MSH and TGF-β2 were injected i.v. with OVA antigen-primed Th1 cells. The results are shown in FIG. 13, a bar chart showing DTH response in mice as measured by ear swelling, i.e., change in ear thickness (μm), as a function of the type(s) of T cells administered, including whether or not the mice were injected with activated, OVA-primed T cells treated with α-MSH and TGF-β2 (“Regulatory T cells; α-MSH/TGF-β2 OVA”). α-MSH/TGF-β2-treated T cells suppress DTH mediated by other, responder T cells (Th1). Activated OVA-primed T cells treated with α-MSH and TGF-β2 (Regulatory T cells; α-MSH/TGF-β2 OVA) were injected i.v. with DTH-mediating T cells (Responder T cells; OVA). OVA-pulsed APC were injected into the ear pinna and ear swelling was measured 24 hours later. The data is representative of two experiments with similar results and is presented as the percent difference in ear thickness±SEM (n=5). P=0.05.

FIG. 13 shows that α-MSH—and TGF-β2—treated, primed T cells suppressed the DTH mediated by the Th1 cells in the pinna of mouse ears injected with OVA pulsed APC. Therefore, α-MSH in conjunction with TGF-β2 mediated activation of functional regulatory T cells, generally known as Th3 cells.

Aqueous humor and its immunosuppressive factors α-MSH and TGF-β2 mediated induction of Th3 cells. These Th3 cells proliferated and produced TGF-β.^{A8} The induced Th3 cells suppressed Th1 cells from mediating DTH. Our results suggest that the ocular microenvironment has the potential to locally divert primed T cells that are programmed to have a Th1 response, into a Th3 response when activated. The results are also the first report that specific physiologically relevant factors can mediate induction of Th3 cells. The ability for the ocular microenvironment and for α-MSH and TGF-β2 to mediate induction of Th3 cells has implications on the manner by which an immune response is elicited and regulated in the eye.

TGF-β-producing T cells have been described in the oral tolerance models of experimental autoimmune uveitis (EAU) and encephalomyelitis (EAE)^{A13,A14}. In the oral tolerance models, low doses of orally administered autoantigens induce, through the gut associated lymphoid tissues, (GALT) activation of TGF-β-producing T cells that actively suppress autoimmune disease^{A15}. These regulatory T cells, also known as Th3 cells, suppress the activity of other disease mediating T cells through their secretion of anti-inflammatory mediators^{A13-A15}. Such activity results in tolerance to the autoantigen, defined by reduced inflammation at sites of autoantigen-mediated disease. It is possible that the ocular microenvironment, through its constitutive production of α-MSH and TGF-β2, mediates induction of Th3 cells as a potential mechanism to mediate the peripheral tolerance to ocular antigens^{A16, A17}.

The results indicate that α-MSH is sufficient for induction of regulatory T cells. However, aqueous humor also contains TGF-β2. TGF-β2 itself can also induce activation of regulatory T cells; however, T cell proliferation is relatively suppressed. When TGF-β2 was added to the cultures 4 hours after primed T cells were TCR-stimulated in the presence of α-MSH, there was significant proliferation of, and TGF-β production by, the T cells. Since our primed T cells, when activated, normally function as Th1 cells, the results suggest that α-MSH diverts their Th1 programming into Th3, which diversion is enhanced by TGF-β2. Also, this change requires time to develop within the TCR-stimulated T cells.

In addition, the effects of TGF-β1 and TGF-β2 are different. Regulatory T cell induction does not occur in the presence of TGF-β1. It appears that TGF-β1 clearly suppresses α-MSH induction of regulatory T cell activity. TGF-β1 could be considered under the experimental conditions to be immunosuppressive, while TGF-β2 is immunomodulating. The only possible means that could mediate a differential response to TGF-β1 and TGF-β2 would be through the different receptor requirements for binding the two TGF-β isoforms and the different affinities for TGF-β1 and TGF-β2 by the type II receptor^{18,19}. Therefore, changes in TGF-β receptor signals, possibly influenced by α-MSH, may mediate the different responses seen by activated primed T cells treated with either TGF-β1 or TGF-β2 in the presence of α-MSH.

The finding that TGF-β2 but not TGF-β1 could induce activation of regulatory T cells, is in line with the finding that it is TGF-β2 protein found in aqueous humor^{A2, A5, A7}. Under uveitic conditions where the blood-ocular barrier is compromised, entry of TGF-β1 from plasma into the aqueous humor could antagonize α-MSH and TGF-β2 induction of regulatory T cells. This could promote activation, and if active TGF-β1 is at a low concentration, enhance proliferation of activated uveitis-mediating T cells. Recently it has been found that IL-4 and TGF-β can mediate development of TGF-β-producing T cells from a population of naive T cells^{A11}. Previously we have shown that the effects of α-MSH on primed T cells is similar to the effects of IL-4 on primed T cell activation A10. Therefore, it is possible that we are observing a similar cytokine-mediated mechanism by aqueous humor in the induction of regulatory T cells with α-MSH inducing IL-4-like signals followed by the effects of TGF-β2 in the T cells. This would be similar to primed/memory T cells entering the ocular microenvironment, being influenced immediately by α-MSH and then, in time, encountering cells producing and activating TGF-β2.

The results here demonstrate that aqueous humor and therefore the ocular microenvironment, possibly through α-MSH and TGF-β2, goes beyond suppressing activation of Th1 cells.^{A2,A20} Aqueous humor also promotes activation of Th3 cells. Therefore, only specific types of immunological responses are activated within the normal ocular microenvironment. The induction of regulatory, Th3 cells could reinforce the immunosuppressive ocular microenvironment of the eye by their contribution of immunosuppressive lymphokines and by their suppression of Th1 cell activity. The potential for activating autoreactive Th3 cells suggests that their presence in vivo could prevent or eliminate clonal expansion and activation of disease-mediating autoimmune Th1 cells.

The ability for the ocular microenvironment to produce constitutive levels of immunosuppressive cytokines that also mediate induction of Th3 cells is an example of how a regional tissue site can manipulate, mold, and coerce an immune response that is tailored for the needs of the tissue. The eye's use of cytokines to regulate the immune response allows for examining the possibility whether these specific immunoregulating factors are neutralized, antagonized, or no longer produced in eyes that are susceptible to or suffering from autoimmune uveitis. It may even be possible to use the same factors to systemically and locally manipulate the immune response to suppress immune-mediated inflammatory diseases. The finding that the ocular microenvironment may induce activation of Th3 cells is an indication that the induction of such regulatory T cells may be a normal physiological occurrence within the eye and that failure of the ocular microenvironment to maintain induction of autoreactive Th3 cells could make it susceptible to autoimmune disease.

### Example III

Autoimmune disease is suppressed by injections of α-MSH treated T cells. To examine the possibility that α-MSH can induce regulatory T cells, α-MSH was used to induce autoreactive regulatory T cells that suppress autoimmune disease. The autoimmune disease model examined was experimental autoimmune C27 uveoretinitis (EAU) of B10.RIII mice^{C27}. The EAU is induced by immunizing the mice with the peptide fragment 161-180 of human interphotoreceptor retinoid binding protein (IRBPp) in Freund's adjuvant^{C30}. The retinal inflammation was seen starting 9 days after the immunization, peaked in 15 days, and resolved after 24 days. On the day of immunization the mice were injected with α-MSH treated T cells. The T cells were syngeneic lymph node T cells primed to IRBPp. Prior to being injected, the T cells were antigen-activated in the presence of α-MSH by IRBPp-pulsed antigen presenting cells (APC) for 24 hours. The concentration of α-MSH used in the cultures was the constitutive concentration of α-MSH in mammalian aqueous humor of normal eyes, 30 pg/ml^{C23}. These α-MSH treated T cells were i.v. injected into B10.RIII mice immunized for EAU.

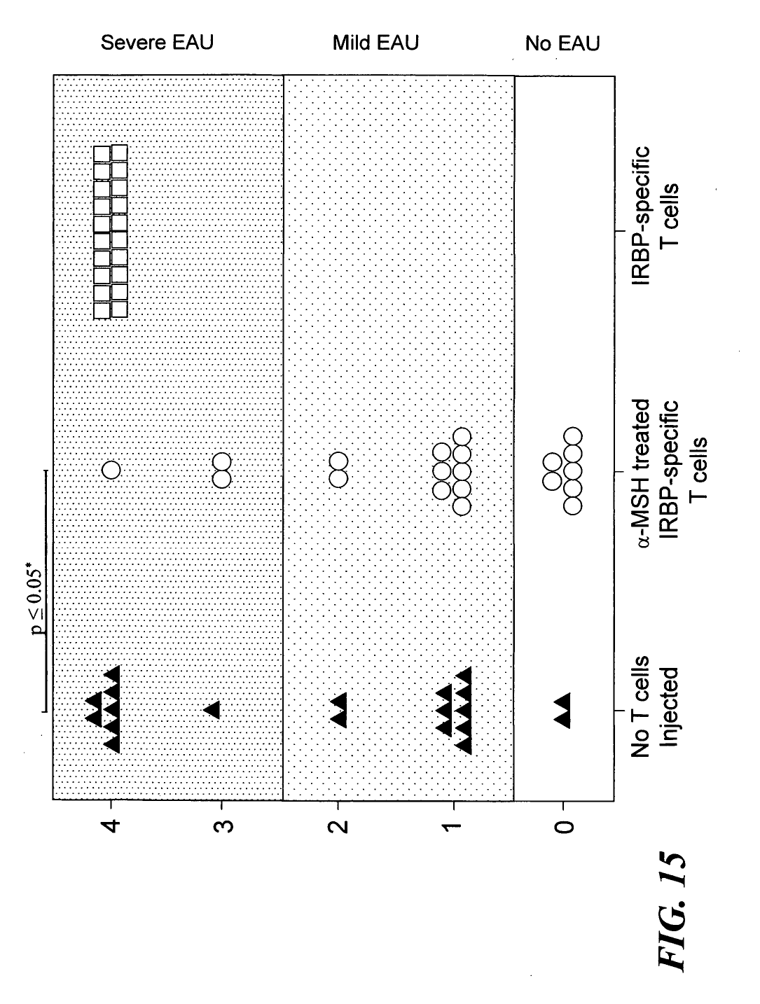

As shown in FIG. 15, B10.RIII mice were immunized with IRBPp to induce EAU on the day 0. On the same day IRBPp-primed T cells (2.5×10⁵ cells/mouse) activated in the presence (open circles) or absence (open squares) of α-MSH were injected into the mice i.v. (as explained in methods). Control EAU mice were not injected with cells (closed triangles). Ocular fundus examinations of the mice were performed every 3 days, and the severity of inflammation was clinically graded on a score 0 to 5. The data presented is the maximum clinical score obtained by each eye in the experimental groups. *The clinical scores are significantly (p≦0.05) different between these two groups as determined a nonparametric Mann-Whitney test for the comparison of two independent populations.

The injection of α-MSH treated T cells specific for IRBPp significantly suppressed both the incidence and severity of EAU (FIG. 15 and Table 1). Moreover, α-MSH appears to have converted autoantigen-reactive T cells from a state that would have further promoted the inflammation of EAU (transfer of IRBPp-specific T cells activated without α-MSH) into regulatory T cells that suppressed expression of autoimmune disease (FIG. 15). This suppressive activity was antigen specific for the ocular antigen, since the transfer of OVA-specific T cells activated in the presence of α-MSH had no significant influence on the course of EAU (Table 1). This last finding corresponds to our previous findings that α-MSH treated effector T cells require the presentation of their specific antigen to expresses suppressive activity^{C29}. The results demonstrate that the use of α-MSH and primed T cells can generate in vitro regulatory T cells. The adoptive transfer of autoantigen specific T cells treated with α-MSH in this manner suggest an ex vivo method for the suppression of autoimmune disease.

TABLE 1
The suppression of EAU by α-MSH-induced
autoreactive-regulatory T cells.

| | | Mean day of | | Day of |
|---|---|---|---|---|
| Injected | | maximum | | resolution |
| into EAU | Mean Maximum | score of the | % | (Score ≦ |
| mice* | Score (±SD)^{†} | group | Incidence^{‡} | 0.5) |
| No T cells | 2.2 ± 1.3 | 15 | 90 | 24 |
| IRBP specific | 4.0 ± 0.0^{§} | 13 | 100 | >24^{#} |
| T cells | | | | |
| α-MSH treated | 1.1 ± 1.3^{§} | 15 | 65^{¶} | 21 |
| IRBP-specific | | | | |
| T cells | | | | |
| α-MSH treated | 1.8 ± 1.3 | 15 | 70 | 24 |
| OVA-specific | | | | |
| T cells | | | | |

*B10.RIII mice (10 per group) were immunized against IRBP with complete
Freund's adjuvant. Within an hour of the immunization, 2 × 10⁵ IRBP or OVA primed-T cells activated 24 hours before in the presence or absence of α-MSH (30 pg/ml) in vitro by IRBPp or OVA- antigen presented adherent spleen cells.
^{†}Mean maximum clinical score was calculated from 20 eyes (10 mice) within each treatment group.
^{‡}Incidence is the percentage of eyes that reached at least a clinical score of 1 anytime in the course of the expreiments.
^{§}Significantly (p ≦ 0.05) different from mice that received no injection of T cells as determined by a non-parametric Mann-Whitney test for comparison of two independent populations.
^{¶}Significantly (p ≦ 0.003) different from mice that received no injection of T cells determined by Two sample Z-test of proportions.
^{#}The severity of the uveoretinitis continued beyond the length of the experiment at levels between scores 1 and 2.

α-MSH induces TGF-β producing regulatory T cells. Since T cells are defined by their lymphokine profile, and if α-MSH mediates induction of regulatory T cells, there should also be a distinct pattern of lymphokines produced by these effector T cells. To examine the production of lymphokines, a T cell culture assay was used to detect the effects of α-MSH on T cells alone. Primed T cells isolated from draining lymph nodes of immunized BALB/c mice were stimulated with anti-TCR antibody 2C11 in the presence of α-MSH under serum-free conditions^{C23}.

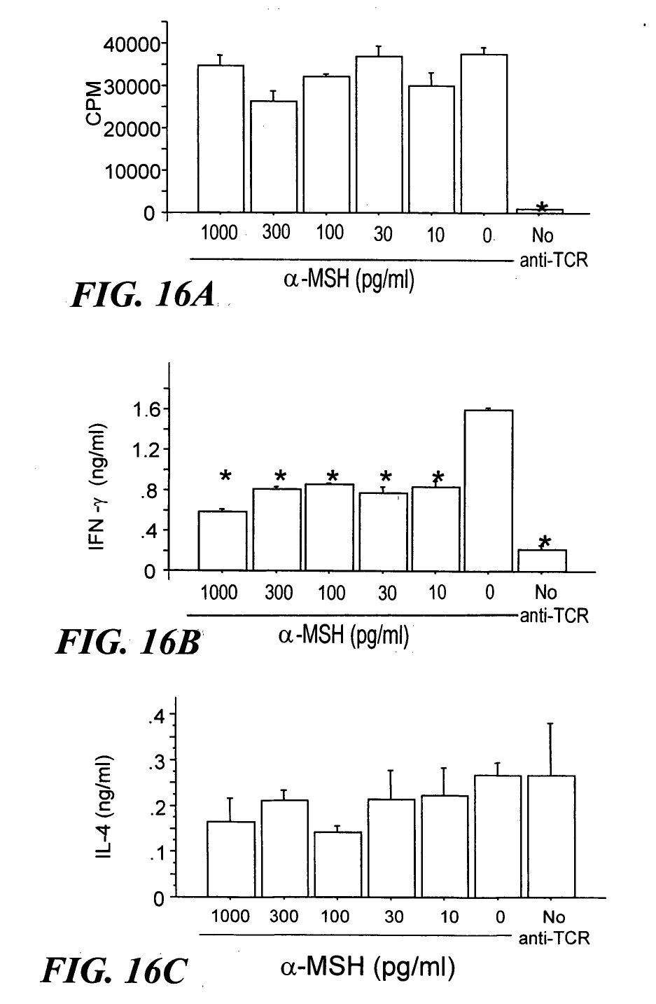

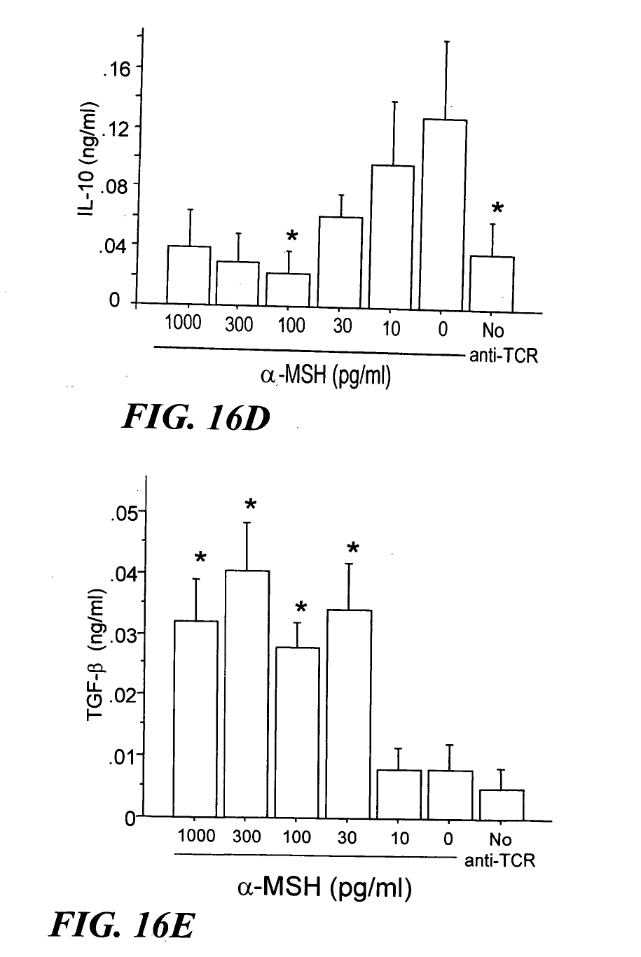

As shown in FIG. 16, α-MSH mediates induction of TGF-β producing regulatory T cells. In FIG. 16A, proliferation was measured by adding to the T cell cultures 3H-thymidine 24 hours after activation and measuring the counts per minute (CPM) 24 hours later. In FIG. 16(B-D), lymphokine production was measured by assaying the 48 hour culture supernatants by sandwich ELISA for IFN-γ, IL-4, and IL-10. In FIG. 16E, to assay for TGF-β, the 48 hour supernatants of the T cell cultures were transiently acidified and assayed by bioassay for total TGF-β. *Significantly different (p≦0.05) from cultures of T cells treated with only anti-TCR (α-MSH at 0 pg/ml). The results are presented as CPM or ng/ml±SEM of four independent experiments.

As expected α-MSH had no affect on the TCR stimulated proliferation (FIG. 16A), but significantly suppressed IFN-γ production by the activated effector T cells (FIG. 16B). Interestingly, α-MSH had no effect on detectable IL-4 levels, which did not change following TCR stimulation, but suppressed IL-10 production in the cell cultures (FIGS. 16C and 16D). This suggests the possibility that unlike other defined regulatory T cells, α-MSH induced regulatory T cells may function in a manner independent of IL-4 and IL-10 production.

The most striking effect of α-MSH on effector T cell lymphokine was α-MSH induced TGF-β1 production by the activated T cells (FIG. 16E). There appeared to be a threshold of α-MSH concentration needed to induce TGF-β1 production by the TCR-stimulated T cells. This threshold was right at the physiological concentration of α-MSH (30 pg/ml) in immune privileged tissues. These results demonstrate that α-MSH selectively modulates the production of lymphokines by activated effector T cells. This modulation suppresses production of pro-inflammatory lymphokines in favor of lymphokines that regulate immunity. Therefore, α-MSH mediates the induction of effector T cells that produce TGF-β and some IL-4, which is characteristic of regulatory (suppressive) T cells^{C31}.

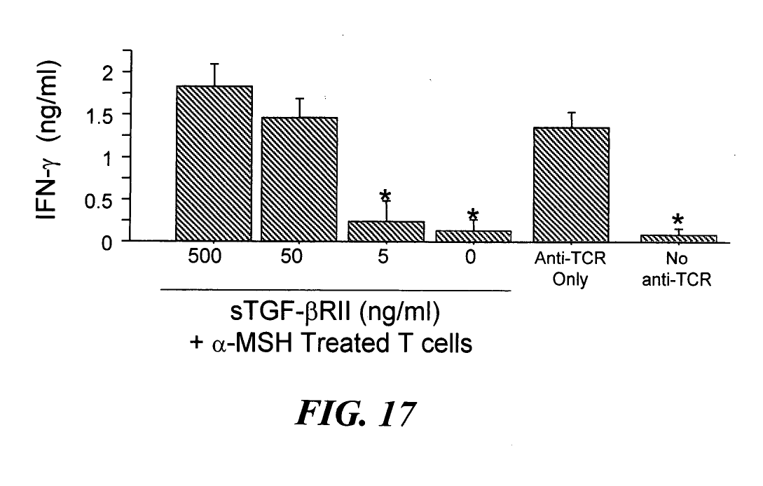

α-MSH induced regulatory T cells that through TGF-β suppress activation of other T cells. To demonstrate that the regulatory T cells induced by α-MSH through their production of TGF-β1 suppress the activation of other T cells, an in vitro regulatory T cell assay was used as previously reported^{C29}. As shown in FIG. 17, the regulatory T cells were generated by activating primed T cells in the presence α-MSH (30 pg/ml) for 48 hours. The regulatory T cells were washed and added to cultures of freshly TCR-stimulated primed T cells as described for FIG. 15. To these cocultures soluble-TGF-β receptor type II (sTGF-PRII) was added. The culture supernatant was assayed 48 hours later by sandwich ELISA for IFN-γ. *Significantly different (p≦0.05) from cultures with only anti-TCR added. The results are presented as IFN-γ±SEM of four independent experiments.

The freshly activated primed T cells produce significant levels of IFN-γ without the addition of α-MSH treated T cells (FIG. 17). By contrast there was a significant reduction in IFN-γ levels when the α-MSH treated T cells were added to the cultures FIG. 17, no sTGF-βRII). Therefore, the addition of T cells treated with α-MSH suppressed the activation of the freshly TCR-stimulated primed T cells. To demonstrate the role of TGF-β in the suppressive activity mediated by the regulatory T cells, TGF-β activity was neutralized with soluble TGF-β receptor type II (sTGF-βRII) added to the mixed T cell cultures (FIG. 17). The suppression mediated by the α-MSH-treated T cell of IFN-γ production by other activated effector T cells was neutralized with the addition of increasing amounts of sTGF-βRII to the cell cultures. The addition of sTGF-βRII permitted activation of the primed T cells. The α-MSH-treated T cells had through the activity of TGF-β suppressed the production of IFN-γ by other effector T cells.

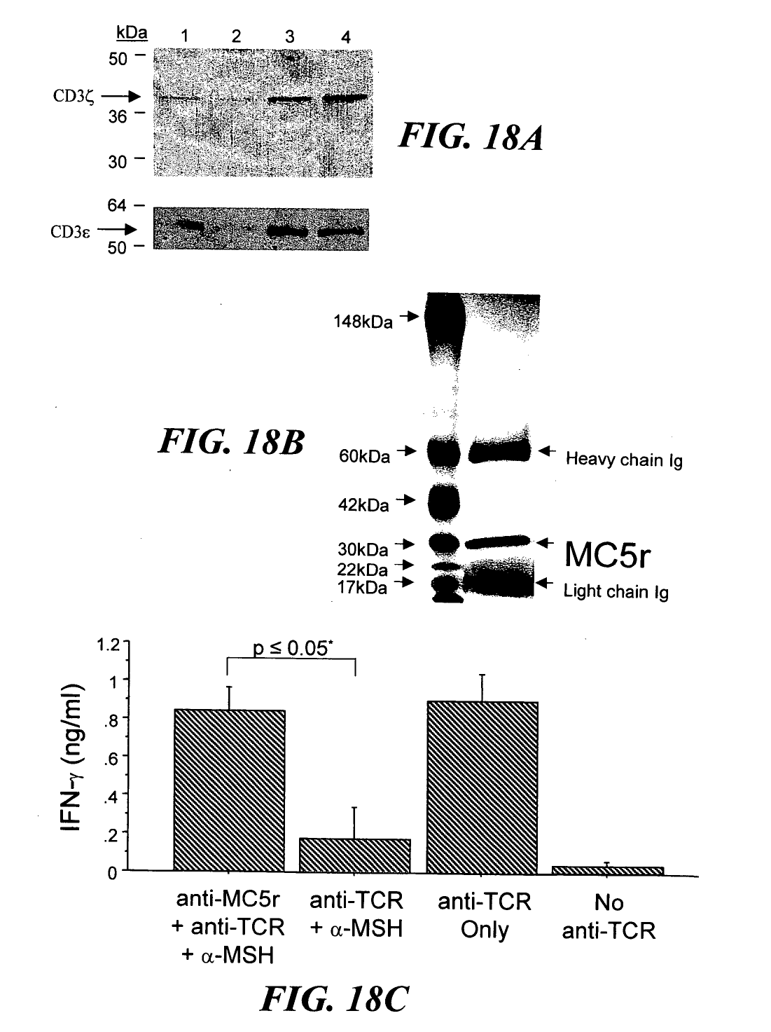

Mouse effector T cells express melanocortin 5 receptor (MC5r). To see if the α-MSH-mediated differential activation of lymphokine production was through a change in the immediate TCR-activation signals of T cells, the intensity of tyrosine phosphorylation of CD3ζ and ε chains was examined. As shown in FIG. 18, (A) T cells enriched from primed lymph nodes were activated with anti-TCR antibody 2C11 in the presence or absence of α-MSH (30 pg/ml). T cells were lysed 15 minutes after the start of the cultures. The lysates were immunoprecipitated with anti-CD3ζ or anti-CD36 antibodies. The precipitants were electrophoresed (8-16% gradient gel) and transferred to a nitrocellulose membrane. The nitrocellulose was incubated with anti-phosphotyrosine antibody. Lane 1) Unstimulated T cells with α-MSH; Lane 2) Unstimulated T cells; Lane 3) activated T cells; Lane 4) T cells activated in the presence of α-MSH. (B) Primed T cells were activated in the presence of α-MSH and lysed 15 minutes later. Lysate was electrophoresed (8% gel) and transferred to nitrocellulose. The nitrocellulose was probed with anti-MC5r antibody. (C) T cells enriched from primed lymph nodes were activated with anti-TCR antibody 2C11 in the presence of α-MSH (30 pg/ml) and anti-MC5r antibody (1 μg/ml). The cultures with only anti-TCR also contained an irrelevant goat IgG (1 μg/ml). The culture supernatants were assayed 48 hours later by sandwich ELISA for IFN-γ. The results are presented for the concentration of IFN-γ (ng/ml)±SEM of four independent experiments. *Significance (p≦0.05) was determined between α-MSH treated T cells and α-MSH treated T cells with anti-MC5r antibody added to the culture.

Primed T cells activated in the presence of α-MSH had no significant change in the expected overall level of tyrosine phosphorylation of CD3ζ and ε molecules from TCR-stimulated T cells (FIG. 18A). Therefore, α-MSH must be initiating signals that are further down-stream of the immediate TCR-activation signals and possibly separate from the TCR-signals that initiate T cell proliferation.

The most likely mechanism of α-MSH mediating lymphokine production is through its own receptors expressed on lymphocytes. The α-MSH binding receptors are generically considered G-protein-coupled melanocortin receptors (MCr)^{C32}. In humans there are five known MCr of which all but MC2r bind α-MSH. Very little is known about the expression of MCr in mice. Recently, MC5r has been characterized on mouse B cells and rat splenic lymphocytes^{C33,C34}. Unique to MC5r is its link to intercellular JAK 2 to STAT 1 and STAT 2 signal pathways^{C33}. It is through this receptor that a-MSH can mediate proliferation by the B cells through similar intracellular signal transducing pathways as other cytokines and growth factors. To see if this characterized receptor is also expressed on the effector mouse T cells, immunoprecipitates of lysed primed T cells were immunoblotted with anti-MC5r antibody. FIG. 4B reveals the presence of the 32 kDa MC5r protein from primed mouse T cells. Moreover, addition of anti-MC5r antibody to cultures of effector T cells activated in the presence of α-MSH neutralized α-MSH suppression of IFN-γ production (FIG. 18C). Therefore, T cells express at least MC5r and that it is linked to α-MSH regulation of lymphokine production.

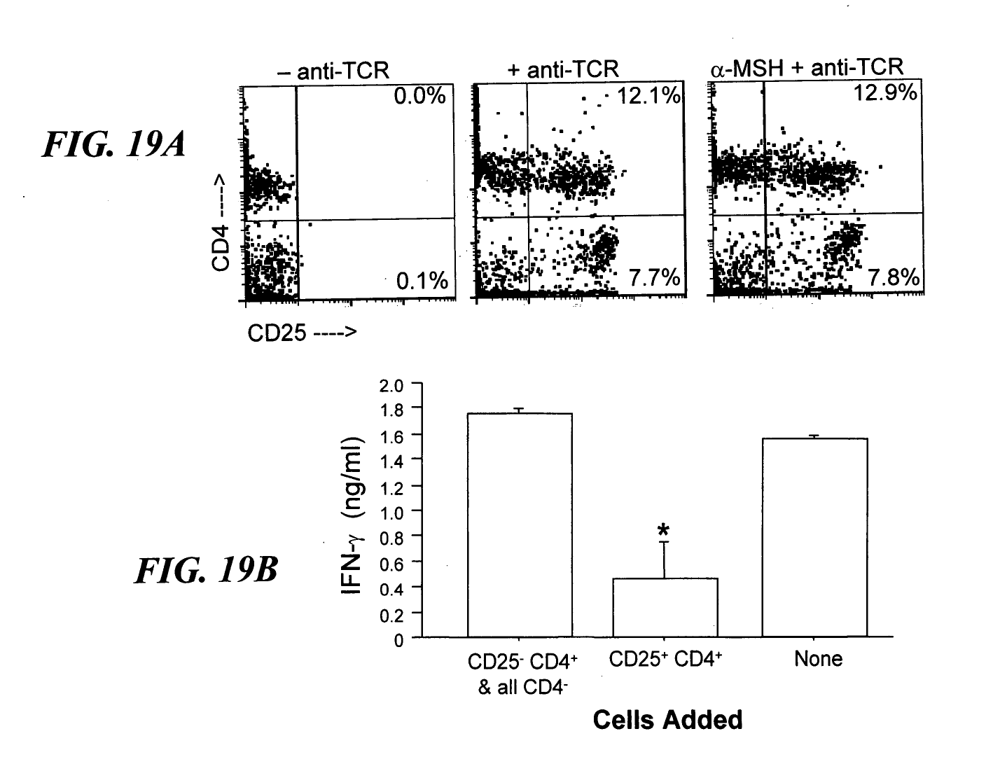

α-MSH induced CD25⁺ CD4⁺ regulatory T cells. To characterize the regulatory T cells induced by α-MSH, we assayed through flow cytometry the possibility that α-MSH induces CD25⁺ (IL-2Rα) CD4⁺ regulatory T cells. As shown in FIG. 19, (A) T cells enriched from primed lymph nodes were activated with anti-TCR antibody 2C11 in the presence or absence of α-MSH (30 pg/ml). The cells were analyzed by two-color flow cytometry after staining with anti-CD4 and anti-CD25. Presented is a representative dot plot of the flow cytometry seen in four separate staining experiments. (B) Only T cells activated in the presence of α-MSH, were stained and sorted based on the coexpression of CD4 and CD25. The sorted cells were added to cultures of freshly activated primed T cells and tested for regualtory activity as in FIG. 3. The supernatants of the cocultures were assayed 48 hours later by sandwich ELISA for IFN-γ. *Significantly (p≦0.05) different from cultures with no additional T cells (None). The data are present as IFN-7 (ng/ml)±SEM of four independent experiments.

The primed T cells were activated by TCR-stimulation in the presence of α-MSH (30 pg/ml) and incubated for 24 hours. The cells were stained for CD25 and CD4. The percentage of CD25⁺ CD4⁺ cells did not change between cultures of primed T cells TCR-stimulated in the presence or absence of α-MSH (FIG. 19A). However, it is the CD25⁺ CD4⁺ T cells that emerge following α-MSH treatment that are the regulatory T cells (FIG. 19B). The CD25⁺ CD4⁺ T cells were sorted by flow cytometry and added to cultures of freshly activated primed T cells in the in vitro regulatory T cell assay. It is this population of T cells alone that suppressed IFN-γ production by other primed T cells. Addition of CD25⁺ CD4⁺ T cells from the T cells that were not treated with α-MSH did not affect the IFN-γ production by the freshly-activated primed T cells (data not shown). In the presence of α-MSH there is induction of CD25⁺ CD4⁺ regulatory T cells.

The preceding results demonstrate that the neuropeptide α-MSH mediates the induction of TGF-β-producing T cells. The lymphokine profile of α-MSH-treated primed T cells is immunosuppressive instead of pro-inflammatory. Therefore, the suppression of autoimmune uveitis appears to be due to α-MSH suppressing production of inflammatory lymphokines by activated autoreactive T cells while inducing activation of regulatory T cells. The Example II results demonstrated that such α-MSH-treated T cells suppress antigen-specific DTH in vivo through by-stander suppression. This observation suggested that α-MSH regulation is mediated through non-antigen specific mechanisms, such as a cytokine. The results shown in Tables 1 and 2 demonstrate that it is at least through the production of TGF-β that α-MSH-treated T cells mediate immunosuppression. Therefore, α-MSH induces activation of regulatory T cells that can mediate regional immunosuppression by producing soluble immunosuppressive factors.

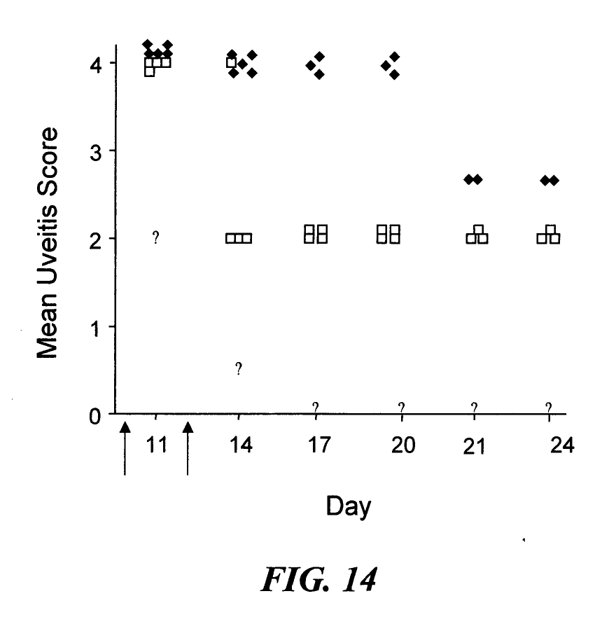

These results also indicate that if the concentration of α-MSH is sufficiently elevated either systemically or regionally, then immunogenic inflammation is suppressed. Along with mediating the suppression of inflammatory T cells and inducing immunosuppressive lymphokine production, α-MSH also directly antagonizes IL-1, TNF, and IFN-γ-inflammatory activities. In addition, the lymphokines produced by the α-MSH-induced, CD25+/CD4+ regulatory T cells have the potential to suppress inflammatory macrophage activities.^{B34,B35} Therefore, the suppression of autoimmune disease seen in FIG. 14 probably results from the general anti-inflammatory activity of α-MSH (i.e., against innate immunity), along with the effects of α-MSH on activated T cells. There is also the potential that regulatory T cells have been induced by the systemic injections of α-MSH.

The results presented here further suggest that α-MSH has a physiological role in regulating inflammatory immune responses. Its activity within a localized tissue site can regulate the intensity and duration of a T cell-mediated inflammatory response, and through induction of regulatory T cells, can also affect whether immunogenic inflammation can occur at all (i.e. induce tolerance). In immune-privileged eyes, there is normally a constitutive presence of α-MSH.^{B7} Since the ocular microenvironment has adapted several mechanisms to prevent induction of inflammation, α-MSH may potentially affect immune cells in the eye more so than in other tissue sites. It is likely that within the normal ocular microenvironment, α-MSH mediates induction of TGF-β-producing, CD25+/CD4+ regulatory T cells that in turn mediate peripheral tolerance to ocular autoantigens. Therefore, the ability of α-MSH to selectively regulate the expression of lymphokines in activated T cells means that α-MSH can regulate the induction, intensity, and type of immune response that occurs in a regional tissue site.

The suppression of DTH by adoptive transfer of α-MSH-induced, antigen-primed regulatory T cells, shown in Example II, suggests that such regulatory T cells, if primed by an autoantigen, would suppress induction of inflammatory, T-cell-mediated autoimmune disease. Such autoreactive, regulatory T cells would only be activated in sites where their autoantigen is presented. Through their production of immunosuppressive lymphokines, these CD25+/CD4+, TGF-β-producing T cells would down-regulate or suppress the activation of nearby, autoreactive inflammatory T cells. Such regulation could occur either in the periphery or within a draining lymph node. It is to be seen where α-MSH-induced regulatory T cells migrate in vivo.

There are several reports describing different types of CD25⁺ regulatory T cells. One described population of regulatory T cells are the CD25⁺ CD4⁺ T cells found in the blood circulation of normal healthy mice^{C35-C37}. Their origin is the thymus. The depletion of these cells through thymectomy results in organ specific autoimmune diseases^{C37}. The adoptive transfer of CD25⁺ CD4⁺ T cells into thymectomized mice suppresses the autoimmune diseases. These regulatory T cells required TCR-stimulation to mediate suppression^{C36}. Others have described CD25⁺ CD4⁺ regulatory T cells that require activation with costimulation either through CD28 or CTLA-4^{C38-C40}. Some have also reported that regulatory T cells have characteristics of memory T cells^{C41}. In addition, maintenance of some autoantigen-specific CD25⁺ CD4⁺ T cells is dependent on the presence of the organ containing the autoantigen^{C42}. This suggests that in their development some the CD25⁺ CD4⁺ regulatory T cells must continuously encounter their antigen in the periphery. It appears from the literature there may be several lineages of CD25⁺ CD4⁺ regulatory T cells with as many mechanisms to induce their development and activation.

The regulatory T cells induced by α-MSH have similar characteristics of some of the already described CD25⁺ CD4⁺ T cells and are like the regulatory T cells induced in oral tolerance^{C31,C43,C44}. The cytokine profile of the α-MSH induced regulatory T cells is suppressed in IFN-γ and IL-10 production, but enhanced TGF-β1 and no change in IL-4 production. This is a similar lymphokine profile that has been described for a subset of Th3 cells^{C31}. However, unlike the Th3 cells induced in oral tolerance^{C31,C43,C44}, the regulatory T cells induced by α-MSH suppress other effector T cells through TGF-β1. This gives the regulatory T cells a non-specific mechanism of suppression like the thymic derived regulatory T cells except the α-MSH-induced regulatory T cells require specific-antigen to activate their suppressive activity. The regulatory T cells induced by α-MSH are derived from a population of T cells that have already experienced antigen. They are derived from a population of T cells initially primed to mediate inflammation. It still remains to be seen if the regulatory T cell function mediated by α-MSH results from α-MSH mediating a selective activation of lymphokines; that is converting a type-1 polarized population into a type-3 polarized T cell population. It is clear that α-MSH suppresses activation of inflammatory T cells while promoting the activation of regulatory T cells. The overall result is α-MSH mediating immunosuppression, and possibly tolerance, when the regulatory T cells are adoptively transferred in vivo.

The possibility that a specific cytokine can mediate induction of CD25⁺ CD4⁺ regulatory T cells may lead to understanding the molecular mechanisms involved in the activation and functionality of regulatory T cells. For α-MSH to affect T cell function we observed two features; 1) the T cells must be stimulated through the TCR, and 2) the T cells must express MC5r. Since we could not observe a change in the overall phosphorylation of the CD3 molecules, it is most likely that the effects of α-MSH are downstream from the initial induction of signal from the engaged TCR. What is interesting is that we detect the expression of MC5r by primed T cells. This receptor has been found to be expressed by mouse B cells and is linked to intracellular activity of JAK2 and the phosphorylation and migration to the nucleus of STAT1 and STAT2 nucleotide binding proteins^{C33}. This indicates that through MC5r, α-MSH can mediate intracellular events that are associated with cytokine receptor activation, suggesting that the in vitro effects of α-MSH could be a form of cytokine-mediated lymphocyte development. We demonstrate that suppression of IFN-γ production by α-MSH is dependent on the engagement of α-MSH to the MC5r. Since the suppression of IFN-γ production is a necessary step toward development of regulatory T cells, the suppression of type 1 responses, α-MSH could be mediating a differential lymphokine expression by the T cells in a manner that is seen with IL-4 suppression of IFN-γ, and IFN-γ to IL-4 production^{C45}.

α-MSH mediates different T cell functionalities primarily through the engagement of MC5r receptor. No MC3r was found in primed T cells, and we have not directly tested for the expression of MC1 and MC4r. MC4r is highly unlikely to be expressed by T cells since its distribution is restricted to the brain, especially to the sites regulating metabolism^{C46,C47}. Each of the four receptors bind selectively to either the C- or the N-terminus of α-MSH. MC5r and MC3r require binding to both the N- and the C-terminus of α-MSH for function^{C48}. For MC1r and MC4r the C-terminus was sufficient for activation of receptor function. The C-terminal tripeptide of α-MSH has been found to mediate the suppression of inflammatory macrophages, which express in abundance MC1r^{C1,C4}. This indirectly suggests that MC1r is not involved in the immunomodulating activity observed for α-MSH. The results do indicate that the intracellular events initiated by α-MSH engagement with its receptor MC5r must intersect with the intracellular events initiated by the engagement of the TCR resulting in the stable expression of regulatory activity by the effector T cells.

The immunomodulating activity of α-MSH suggests that it can be used to suppress autoimmune disease. Direct injections of α-MSH into eyes of mice with EAU can suppress the severity of the inflammation and accelerate recovery^{C28}. It has been discovered that injections of α-MSH treated T cells that are antigen-specific for the ocular autoantigen can also suppress the incidence and severity of EAU in mice. Therefore, α-MSH can antagonize an ongoing autoimmune disease either directly or indirectly through the ex vivo induction of regulatory T cells by α-MSH. Therefore, it may be possible to manipulate a tissue microenvironment to suppress immunogenic inflammation and induce the activation of regulatory T cells that may mediate tolerance to the target tissue antigen. In addition, this ability of α-MSH to manipulate immunity is an extreme example of the interactions between the neuroendocrine and immune systems and is one that is found within immune privileged tissues^{C25}.

The present work has demonstrated the importance of the neuropeptide α-MSH in regulating the adaptive immune response, i.e., inflammation mediated by T cells. α-MSH selectively regulates the production of lymphokines by activated effector T cells. These effector T cells display enhanced levels of TGF-β1 production and no IFN-γ or IL-10 with IL-4 levels remaining unchanged in comparison with inactivated T cells.

Additionally, if soluble TGF-β receptor II was added to co-cultures of α-MSH treated T cells and activated Th1 cells, the α-MSH treated T cells could not suppress IFN-γ production by the Th1 cells. These results suggest that α-MSH induces T cells with a regulatory lymphokine pattern, and that through their production of TGF-β1 they suppress other effector T cells. Examination of the α-MSH treated T cells showed that α-MSH did not alter the phosphorylation of CD3 molecules following TCR engagement. Primed T cells express the melanocortin 5 receptor (MC5r), a receptor that is linked to an intracellular signalling pathway shared by other cytokine receptors. Blocking the receptor with antibody prevented α-MSH from suppressing IFN-γ production by the activated regulatory T cells, suggesting that α-MSH immunoregulation is through the MC5r on primed T cells. Surface staining and cell sorting of the α-MSH treated primed T cells showed that the regulatory T cells are CD25⁺ CD4⁺ T cells. From these results we find that α-MSH can mediate the induction of CD25⁺ CD4⁺ regulatory T cells. These regulatory T cells require specific antigen for activation but through non-specific TGF-β1-mediated mechanisms they can suppress other effector T cells.

This selective immunoregulation by α-MSH has an important role in maintaining immunogenic homeostasis through suppression of inflammation (both innate and T cell-mediated) and possibly through tolerance of autoantigens. It also supports the use of α-MSH's immunosuppressive activities to treat autoimmune diseases.

### Uses

As supported by the preceding experimental results and discussions thereof, the invention encompasses a method for generating antigen-specific regulatory T cells that can down-regulate or suppress adaptive immune-mediated inflammation, namely inflammatory responses mediated by activated, primed effector T cells generally of the Th1 subclass. In particular, the method generates regulatory T cells that have a CD4⁺/CD25⁺ phenotype and that produce Transforming Growth Factor β (TGF-β), which suggests that they have Th3 cell characteristics.

In one aspect, the regulatory T cell-generating method comprises exposing CD3-enriched, primed T cells to a specific, presented antigen in the presence of antigen-presenting cells (APC) and the presence of a composition comprising an effective amount of alpha-Melanocyte Stimulating Hormone (α-MSH) or an α-MSH receptor-binding portion thereof. In another aspect, the regulatory T cell-generating method comprises exposing CD3-enriched, primed T cells to a T cell receptor-crosslinking agent in the presence of a composition comprising an effective amount of alpha-Melanocyte Stimulating Hormone (α-MSH) or an α-MSH receptor-binding portion thereof.

The α-MSH receptor-binding portion comprises lysine-proline-valine, which represent amino acid residues 11-13 of α-MSH. An effective amount of α-MSH or an α-MSH receptor-binding portion thereof is an amount sufficient to produce an in situ concentration of intact α-MSH in the range of about 20-100 pg/ml, preferably about 30 pg/ml or a molar equivalent amount of an α-MSH receptor binding portion of α-MSH, in the immediate vicinity of the primed T cells during the first exposing step.

Either method optionally further comprises, approximately 4-6 hours after the first exposure step has begun (i.e., the exposure of the primed T cells to a T cell activation signal in the presence of α-MSH or binding portion thereof), exposing the primed T cells to an effective amount of TGF-β2. The TGF-β2 enhances the α-MSH's induction of TGF-β-producing, CD4+/CD25+, regulatory T cells. An effective amount of TGF-β2 is an amount sufficient to produce an in situ concentration in the range of about 1-10 ng/ml, preferably about 5 ng/ml, in the immediate vicinity of the primed T cells.

The composition comprising α-MSH or a binding portion thereof, may further include TGF-β2 in a timed-release delivery vehicle designed to release the TGF-β2 approximately 4-6 hours after the start of the incubation of the primed T cells with the T cell activation signal (e.g., APC-presented antigen or TCR-crosslinking agent) in the presence of the α-MSH-comprising composition.

The “specific antigen” used in the invention is an antigen recognized by the CD3-enriched, primed T cells, and should be one that is presented to the T cell by an antigen-presenting cell.

In general, primed T cells are understood to be T cells that have previously been exposed to a specific antigen under conditions producing at least one “armed” or “memory” T cell clone or subset that specifically recognizes that antigen and mounts an immune response triggered by engagement of the T cell receptor with that antigen as presented by an antigen-presenting cell. Primed T cells can be derived in vivo, by harvesting them from an animal immunized with the specific antigen. Primed T cells can also be produced in vitro, by methods well known in the art, such as culturing naive T cells in vitro with the specific antigen and with other lymphocytes, antigen presenting cells, cytokines, in culture conditions known to stimulate or generate memory effector T cells that specifically recognize and respond to that antigen. Alternatively, “primed T cells” can be stimulated by crosslinking of the T cell receptors by antibodies (e.g., anti-CD3) or T cell mitogens, such as Concanavalin-A (Con-A), Pokeweed mitogen (PWM), or Phytohemagglutinin (PHA).

Therefore, the regulatory T cell-generating method can also include, prior to exposing primed T cells to α-MSH and/or TGF-β2, stimulating the T cells with an antigen and incubating T cells with an anti-T cell receptor antibody or a T cell mitogen to activate the primed T cells.

Generation of regulatory T cells according to the invention, may be done by culturing primed T cells, the specific antigen, α-MSH (with or without later addition of TGF-β2), and appropriate T-cell culture media in vitro. Alternatively, generation of antigen-specific regulatory Th3 cells may be achieved by in vivo exposure of primed T cells to the specific antigen in the presence of α-MSH, with or without addition of TGF-β2 approximately 4-6, preferably about 4, hours after the start of exposure of the primed T cells to the specific antigen and the α-MSH. For instance, a composition comprising at least α-MSH, preferably both α-MSH and TGF-β2, and the specific antigen to be recognized by the desired TGF-β-producing, CD4+/CD225+, regulatory T cells, may be introduced (e.g., by injection or surgical implantation) into an animal previously immunized with that same specific antigen. (Hence, the immunized animal will have T cells primed to that antigen.) Preferably, the antigen and α-MSH-containing composition are introduced into a localized tissue site (e.g., within an eye, a brain, or a transplant site). Alternatively, antigen-primed T cells may be introduced into the animal along with the specific antigen and a composition comprising α-MSH or α-MSH plus TGF-β2.

Another aspect of the invention encompasses a method for down-regulating or suppressing an T cell-mediated inflammation, such as in an autoimmune or a graft rejection response, particularly in a localized tissue site in an animal. Specifically, this method comprises the following steps conducive to generating regulatory Th3 cells, i.e., CD4⁺/CD25⁺ T cells that produce TGF-β2:
(a) harvesting T cells from the animal;
(b) producing primed T cells by exposing the harvested T cells in vitro to a specific antigen under conditions enabling stimulation of at least one memory T cell that specifically recognizes said antigen;
(c) exposing the primed T cells in vitro to a specific antigen in the presence of a composition comprising alpha-Melanocyte Stimulating Hormone (α-MSH), and in the presence of at least one T cell activating factor, namely a T cell receptor-crosslinking agent (e.g., an anti-TCR antibody or a T cell mitogen); and
(d) introducing into an animal (e.g., by injection or implantation), the T cells generated from step (c) (which comprise CD4+ CD25+ regulatory T cells)

The methods for generating regulatory Th3 cells and of regulating T cell-mediated inflammation, may be used to treat autoimmune disorders, e.g., autoimmune uveitis, in humans and other animals, such as. The methods of the invention may also be used in conjunction with transplantation, to suppress or to keep in check, host immune responses responsible for graft rejection.

In all methods of the invention, the α-MSH-containing composition comprises α-MSH preferably in a concentration lying within the range of about 30-100 pg/ml. A preferred embodiment of the method uses λ-MSH in a sufficient concentration to provide an in situ concentration of at least about 30 pg/ml in the localized tissue site in which generation of regulatory Th3 cells is desired, i.e., in the immediate vicinity of the α-MSH-treated, primed T cells. For instance, in the case of treating a self-contained, small site, e.g., an eye, it may be sufficient to use a composition comprising α-MSH in a concentration of about 30 pg/ml.

When primed T cells are exposed to the specific antigen and a composition comprising both α-MSH and TGF-β2, the TGF-β2 is present in the composition in a timed-release delivery vehicle, preferably in a concentration within the range of about 1-10 ng/ml. More preferably, TGF-β2 is used in a concentration effective to achieve a final concentration of about 5 ng/ml within the local environment of the primed T cells.

Conditions suitable for T cell culture are well-known in the art. For instance, the conditions could include culturing the α-MSH-treated, primed T cells in T cell culture medium, preferably a substantially serum-free one. The treated T cells are typically incubated at about 37° C., for an incubation period within the range of about 18-24 hours, more preferably about 24 hours. Exemplary conditions may be found in preceding Examples I, II, and III.

The invention also encompasses a kit for generating antigen-specific regulatory T cells, thereby regulating T cell-mediated inflammation, comprising: (a) a specific antigen; (b) α-MSH or α-MSH receptor-binding portion thereof; and (c) an article of manufacture comprising instructions on how to use components (a) and (b) to generate TGF-β-producing, CD4+/CD25+, regulatory T cells. The specific antigen is one to be recognized by the antigen-specific regulatory T cells desired, and for instance, could be a target molecule of an autoimmune disease. The α-MSH or α-MSH receptor-binding-portion thereof is included generally in an amount effective to direct the development primed T cells toward a TGF-β-producing, CD4+/CD25+ phenotype, preferably an amount sufficient to give a final concentration in the range of about 30-100 pg/ml of whole α-MSH or a molar equivalent amount of an α-MSH receptor-binding portion of α-MSH, during exposure of T cells primed to the specific antigen.

The kit may further comprise TGF-β2 in an amount effective to enhance the development of the α-MSH-treated primed T cells into TGF-β-producing, CD4+/CD25+, regulatory T cells.

The invention also provides an α-MSH-based gene therapy for down-regulating or suppressing an autoimmune disorder or to prevent graft rejection in a transplantation recipient. Specifically, the invention encompasses a method for down-regulating a graft rejection response in a graft recipient, comprising:
(a) transfecting a graft tissue or organ with genetic material for expressing α-MSH in said graft; and
(b) implanting the transfected graft from step (a) into a recipient animal.

Another method for down-regulating an autoimmune response in a tissue site in an animal, comprises directly injecting genetic material for expressing α-MSH, into or near an autoimmune-diseased tissue.

Yet another method for down-regulating an autoimmune response in a tissue site in an animal, comprises:
(a) harvesting a tissue sample from the tissue site;
(b) transfecting the harvested tissue sample with genetic material for expressing α-MSH; and
(c) implanting the transfected tissue sample into the animal.

In terms of gene therapy applications, one may also control an autoimmune disorder or suppress host-versus-graft rejection by transfecting a cell with genetic material coding for an antigen that also contains the α-MSH tripeptide of lysine-proline-valine that is involved in binding to the α-MSH receptor. Insertion of such genetic material could mediate both antigen stimulation of the primed T cell and α-MSH-mediated induction of regulatory T cells.

In another aspect of the invention, a synthetic analogue of α-MSH that targets the MC5r receptor exclusively may be used as therapy for mediating regulatory T cells. Exemplary regulation of T cells includes those described above and, for example, suppressing T cell-mediated inflammatory response. Preferably, the therapeutic treatment targets the MC5r receptor exclusively to manipulate T cell functionality while leaving other MC(1-4)ᵣ receptors' dependent pathways and functions unmodified. Therefore, an exemplary synthetic analogue would have the same functional properties as that of α-MSH described herein that exclusively binds to the MC5r receptor. To determine whether the analogue of α-MSH exclusively binds to the MC5r receptor, an ordinary skilled artisan can use standard procedures known in the art. If it does bind other MC(1-4)r receptors, then the analogue must not antagonize nor agonize the other receptors. Other determinative factors include whether the analogue mediates suppression of IFN-γ production by activated effector cells; whether it mediates the activation of regulatory T cells; whether it mediates the phosphorylation of the intracellular signalling molecules STAT1 and STAT2; whether it does not mediate suppression of macrophage NF-κB activation, which is a functional feature of activated MC1r; and whether the analogue has no functional effects on T cells from MC5r(−/−) knockout mouse.

In a further aspect of the invention, an anti-MC5r antibody, a fragment or an analogue thereof which acts as an agonist to only the bound MC5r, may be used for therapeutic treatment, wherein the antibody binds the MC5r receptor and delivers α-MSH. The antibody may comprise an F(ab)₂ fragment to prevent cytolytic (complement fixation, monocyte phagocytosis) targeting of the cells it bind. The antibody may also bind to MC5r on the surface of a lymphocyte that neither antagonizes (neutralizes) nor agonizes (stimulates) the receptor to deliver α-MSH to the cellular receptor. As an example, the antibody or an analogue thereof may be chemically modified to physically attach α-MSH to a region on the antibody that does not change the antibody's specificity for MC5r, but gives α-MSH or its analogue access to the ligand binding site on MC5r. In another example, the gene for the antibody may be modified to code for α-MSH in a specific region of the antibody allowing for expression of the α-MSH gene. As a preferred example, the antibody would be a monoclonal antibody of mouse origin for an experimental study or a humanized antibody for therapeutic use. One of ordinary skill in the art will appreciate that the preceding gene therapy protocols may be practiced using known transfection techniques, including episomal or chromosomal transfection.

### REFERENCES

1. Cher, D. J., and T. R. Mosmann. 1987. Two types of murine helper T cell clone. II. delayed-type hypersensitivity is mediated by Th1 clones. J. Immunol. 138:3688-3694.
2. VanParijs, L., and A. K. Abbas. 1998. Homeostasis and self-tolerance in the immune system: turning lymphocytes off. Science 280:243-248.
3. Taylor, A. W. 1996. Neuroimmuomodulation in immune privilege: role of neuropeptides in ocular immunosuppression. Neuroimmunomodulation 3:195-204.
4. Gajewski, T. F., and F. W. Fitch. 1988. Anti-proliferative effect of IFN-γ in immune regulation. I. IFN-γ inhibits the proliferation of Th2 but not Th1 murine helper T lymphocyte clones. J. Immunol. 140:4245-4252.
5. Gajewski, T. F., J. Joyce, and F. W. Fitch. 1989. Antiproliferative effect of INF-y in immune regulation. III. Differentail selection of Th1 and TH2 murine helper T lymphocyte clones using recombinant IL-2 and recombinant IFN-γ. J. Immunol. 143:15-22.
6. Maggi, E., P. Parronchi, R. Manetti, C. Simonelli, M.-P. Piccinni, F. S. Rugiu, M. De Carli, M. Ricci, and S. Romagnani. 1992. Reciprocal regulatory effects of INF-g and IL-4 on the in vitro development of human Th1 and Th2 clones. J. Immunol. 148:2142-2147.
7. Streilein, J. W., B. R. Ksander, and A. W. Taylor. 1997. Immune deviation in relation to ocular immune privilege. J. Immunol. 158:3557-60.
8. Constant, S. L., and K. Bottomly. 1997. Induction of TH1 and TH2 CD4⁺ T cell responses. Annu. Rev. Immunol. 15:297-322.
9. Taylor, A. W., J. W. Streilein, and S. W. Cousins. 1994. Alpha-melanocyte-stimulating hormone suppresses antigen-stimulated T cell production of gamma-interferon. Neuroimmunomodulation 1:188-194.
10. Lee, T. H., A. B. Lerner, and V. Buettner-Janusch. 1961. The isolation and structure of α- and β-melanocyte-stimulating hormones from monkey pituitary glands. J. Biol. Chem. 236:1390-1394.
11. Learner, A. B., and M. R. Wright. 1960. In vitro frog skin assay for agents that darken and lighten melanocytes. In Methods of Biochemical Analysis, vol. 8. D. Glick, editor. Interscience Publishers, Inc., New York, N.Y. 295-307.
12. Lipton, J. M., and A. Catania. 1997. Anti-inflammatory actions of the neuroimmunomodulator α-MSH. Immunol. Today 18:140-145.
13. Shih, S. T., O. Khorram, J. M. Lipton, and S. M. McCann. 1986. Central administration of α-MSH antiserum augments fever in the rabbit. Amer. J. Physiol. 250:R803-R806.
14. Holdeman, M., O. Khorram, W. K. Samson, and J. M. Lipton. 1985. Fever-specific changes in central MSH and CRF concentrations. Amer. J. Physiol. 248:R125-R129.
15. Martin, L. W., and J. M. Lipton. 1990. Acute phase response to endotoxin: rise in plasma α-MSH and effects of α-MSH injection. Amer. J. Physiol. 259:R768-772.
16. Lipton, J. M. 1990. Modulation of host defense by the neuropeptide α-MSH. Yale J. Bio. Med. 63:173-182.
17. Star, R. A., N. Rajora, J. Huang, R. Chavez, A. Catania, and J. M. Lipton. 1995. Evidence of autocrine modulation of macrophage nitric oxide synthase by α-MSH. Proc. Natl. Acad. Sci. USA 90:8856-8860.
18. Chiao, H., S. Foster, R. Thomas, J. Lipton, and R. A. Star. 1996. α-Melanocyte stimulating hormone reduces endotoxin-induced liver inflammation. J. Clin. Invest. 97:2038-2044.
19. Catania, A., N. Rajora, F. Capsoni, F. Minonzio, R. A. Star, and J. M. Lipton. 1996. The neuropeptide α-MSH has specific receptors on neutrophils and reduces chemotaxis in vitro. Peptides 17:675-679.
20. Daynes, R. A., B. A. Robertson, B.-H. Cho, D. K. Burnham, and R. Newton. 1987. α-Melanocyte-stimulating hormone exhibits target cell selectivity in its capacity to affect interleukin 1-inducable responses in vivo and in vitro. J. Immunol. 139:103-109.
21. Grabbe, S., R. S. Bhardwaj, K. Mahnke, M. M. Simon, T. Schwarz, and T. A. Luger. 1996. α-Melanocyte stimulating hormone induces hapten-specific tolerance in mice. J. Immunol. 156:473-478.
22. Bhardwaj, R. S., A. Schwarz, E. Becher, K. Mahnke, Y. Aragane, T. Schwarz, and T. A. Luger. 1996. Pro-opiomelanocortin-derived peptides induce IL-10 production in human monocytes. J. Immunol. 156:2517-2521.
23. Taylor, A. W., J. W. Streilein, and S. W. Cousins. 1992. Identification of alpha-melanocyte stimulating hormone as a potential immunosuppressive factor in aqueous humor. Curr. Eye Res. 11:1199-1206.
24. Lawrence, D. A. 1991. Identification and activation of latent transforming growth factor β. Meth. Enzymol. 198:327-336.
25. Miller, A., O. Lider, A. B. Roberts, M. B. Sporn, and H. L. Weiner. 1992. Suppressor T cells generated by oral tolerization to myelin basic protein suppress both in vitro and in vivo immune responses by the release of transforming growth factor beta after antigen-specific triggering. Proc. Natl. Acad. Sci. USA 89:421-5.
26. Chen, Y., V. K. Kuchroo, J. I. Inobe, D. A. Hafler, and H. L. Weiner. 1994. Regulatory T cell clones induced by oral tolerance: suppression of autoimmune encephalomyelitis. Science 265:1237-1240.
27. Caspi, R., L. Stiff, R. Morawentz, N. Miller-Rivero, C. Chan, B. Wiggert, R. Nussenblatt, H. Morse, and L. Rizzo. 1996. Cytokine-dependent modulation of oral tolerance in a murine model of autoimmune uveitis. Ann. N.Y. Acade. Sci. 778:315-324.
28. Wang, Z. Y., H. Link, A. Ljungdahl, B. Hojeberg, J. Link, B. He, J. Qiao, A. Melms, and T. Olsson. 1994. Induction of interferon-gamma, interleukin-4, and transforming growth factor-beta in rats orally tolerized against experimental autoimmune myasthenia gravis. Cell. Immun. 157:353-368.
29. Abehsira-Amar, O., M. Gibert, M. Joliy, J. Theze, and D. L. Jankovic. 1992. IL-4 plays a dominant role in the differential development of Tho into Th1 and Th2 cells. J. Immunol. 148:3820-3829.
30. Weiner, H. L. 1997. Oral tolerance: immune mechanisms and treatment of autoimmune diseases. Immunol. Today 18:335-343.
31. Chan, A. C., D. M. Desai, and A. Weiss. 1994. The role of protein tyrosine kinase and protein tyrosine phosphatases in T cell antigen receptor signal transduction. Annu. Rev. Immunol. 12:555-592.
32. Alberola-Ila, J., S. Takaki, J. D. Kerner, and R. M. Perlmutter. 1997. Differential signaling by lymphocyte antigen receptors. Annu. Rev. Immunol. 15:125-154.
33. Seder, R. A., T. Marth, M. C. Sieve, W. Strober, J. J. Letterio, A. B. Roberts, and B. Kelsall. 1998. Factors involved in the differentiation of TGF-β-producing cells from naive CD4⁺ T cells: IL-4 and IFN-γ have opposing effects, while TGF-β positively regulates its own production. J. Immunol. 160:5719-5728.
34. Buggy, J. J. 1998. Binding of α-melanocyte-stimulating hormone to its G-protein-coupled receptor on B-lymphocytes activated the Jak/STAT pathway. Biochem. J. 331:211-216.
35. Mountjoy, K. G., L. S. Robbins, M. T. Mortrud, and R. D. Cone. 1992. The cloning of a family of genes that encode the melanocortain receptors. Science 257:1248-1251.
36. Kersh, E. N., A. S. Shaw, and P. M. Allen. 1998. Fidelity of T cell activation through multistep T cell receptor ζ phosphorylation. Science 281:572-575.
37. Taylor, A. W., and J. W. Streilein. 1996. Inhibition of antigen-stimulated effector T cells by human cerebrospinal fluid. Neuroimmunomodulation 3:112-118.
38. Letterio, J. J., and A. B. Roberts. 1998. Regulation of immune responses by TGF-β. Ann. Rev. Immunol. 16:137-161.
39. Bennett, N. T., and G. S. Schultz. 1993. Growth factors and wound healing: biochemical properties of growth factors and their receptors. Amer. J. Surg. 165:728-737.
40. Holter, W., F. S. Kalthoff, W. F. Pickl, C. Ebner, O. Majdic, D. Kraft, and W. Knapp. 1994. Transforming growth factor-beta inhibits IL-4 and IFN-gamma production by stimulated human T cells. Int. Immunol. 6:469-75.
41. Song, X. Y., M. Gu, W. W. Jin, D. M. Klinman, and S. M. Wahl. 1998. Plasmid DNA encoding transforming growth factor-beta1 suppresses chronic disease in a streptococcal cell wall-induced arthritis model. J. Clin. Invest. 101:2615-21.
42. Roberts, A. B., and M. B. Sporn. 1988. Transforming growth factor β. Adv. Can. Res. 51:107-145.
43. Cromack, D. T., M. B. Sporn, A. B. Roberts, M. J. Merino, L. L. Dart, and J. A. Norton. 1987. Transforming growth factor b levels in rat wound chamber. J. Surg. Res. 42:622-628.
44. Wangoo, A., H. T. Cook, G. M. Taylor, and R. J. Shaw. 1995. Enhanced expression of type 1 procollagen and transforming growth factor-beta in tuberculin induced delayed type hypersensitivity. J Clin Pathol 48:339-45.
45. Taylor, A. W., P. Alard, D. G. Yee, and J. W. Streilein. 1997. Aqueous humor induces transforming growth factor-beta (TGF-beta)-producing regulatory T-cells. Curr. Eye Res. 16:900-8.
A1. Taylor A W. Neuroimmunomodulation in immune privilege: role of neuropeptides in ocular immunosuppression. Neuroimmunomodulation. 1996;3:195-204.
A2. Streilein J W, Cousins S W. Aqueous humor factors and their effects on the immune response in the anterior chamber. Curr Eye Res. 1990;9:175-182.
A3. Kaiser C, Ksander B, Streilein J. Inhibition of lymphocyte proliferation by aqueous humor. Reg Immunol. 1989;2:42-49.
A4. Takeuchi M, Alard P, Streilein J W. TGF-β promotes immune deviation by altering accessory signals of antigen-presenting cells. J. Immunol. 1998;160:1589-1597.
A5. Granstein R, Staszewski R, Knisely T L, et al. Aqueous humor contains transforming growth factor-β and a small (<3500 daltons) inhibitor of thymocyte proliferation. J. Immunol. 1990;144:3021-3026.
A6. D'Orazio T J, Niederkorn J Y. A novel role for TGF-β and IL-10 in the induction of immune privilege. J. Immunol. 1998;160:2089-2098.
A7. Cousins S W, McCabe MM, Danielpour D, Streilein J W. Identification of transforming growth factor-beta as an immunosuppressive factor in aqueous humor. Investi Ophthalmol Vis Sci. 1991;32:33-43.
A8. Taylor A W, Alard P, Yee D G, Streilein J W. Aqueous humor induces transforming growth factor-β (TGF-β)-producing regulatory T-cells. Curr Eye Res. 1997;16:900-908.
A9. Taylor A W, Streilein J W, Cousins S W. Identification of alpha-melanocyte stimulating hormone as a potential immunosuppressive factor in aqueous humor. Curr Eye Res. 1992;11:1199-1206.
A10. Taylor A W, Streilein J W, Cousins S W. Alpha-melanocyte-stimulating hormone suppresses antigen-stimulated T cell production of gamma-interferon. Neuroimmunomodulation. 1994;1:188-194.
A11. Seder R A, Marth T, Sieve M C, et al. Factors involved in the differentiation of TGF-β-producing cells from naive CD4⁺ T cells: IL-4 and IFN-γ have opposing effects, while TGF-β positively regulates its own production. J. Immunol. 1998;160:5719-5728.
A12. Jampel H D, Roche N, Stark W J, Roberts A B. Transforming growth factor-β in human aqueous humor. Curr Eye Res. 1990;9:963-969.
A13. Caspi R, Stiff L, Morawentz R, et al. Cytokine-dependent modulation of oral tolerance in a murine model of autoimmune uveitis. Ann NY Acade Sci. 1996;778:315-324.
A14. Chen Y, Kuchroo V K, Inobe J I, Hafler D A, Weiner H L. Regulatory T cell clones induced by oral tolerance: suppression of autoimmune encephalomyelitis. Science. 1994;265:1237-1240.
A15. Weiner H L. Oral tolerance: immune mechanisms and treatment of autoimmune diseases. Immunol Today. 1997;18:335-343.
A16. Gery I, Steilein J W. Autoimmunity in the eye and its regulation. Curr Opin Immunol. 1994;6:938-945.
A17. Streilein J. Peripheral tolerance induction: lessons from immune privileged sites and tissues. Transp Proc. 1996;28:2066-2070.
A18. Cheifez S, Weatherbee J A, Tsang M L-S, et al. The transforming growth factor-β system, a complex pattern of cross-reactive ligands and receptors. Cell. 1987;48:409-415.
A19. López-Casillas F, Wrana J L, Massagué J. Betaglycan presents ligand to the TGFβ signaling receptor. Cell. 1993;73:1435-1444.
A20. Cousins S W, Trattler W B, Streilein J W. Immune privilege and suppression of immunogenic inflammation in the anterior chamber of the eye. Curr Eye Res. 1991; 10:287-297.
B1. Streilein, J. W. 1999. Immunoregulatory mechanisms of the eye. Prog. Retin. Eye Res. 18: 357-370.
B4. Cousins, S. W., M. M. McCabe, D. Danielpour & J. W. Streilein. 1991. Identification of transforming growth factor-beta as an immunosuppressive factor in aqueous humor. Investi. Ophthalmol. Vis. Sci. 32: 33-43.
B5. Granstein, R., R. Staszewski, T. L. Knisely, E. Zeira, R. Nazareno, M. Latina & D. M. Albert. 1990. Aqueous humor contains transforming growth factor-β and a small (<3500 daltons) inhibitor of thymocyte proliferation. J. Immunol. 144: 3021-3026.
B6. Jampel, H. D., N. Roche, W. J. Stark & A. B. Roberts. 1990. Transforming growth factor-□ in human aqueous humor. Curr. Eye Res. 9: 963-969.
B7. Taylor, A. W., J. W. Streilein & S. W. Cousins. 1992. Identification of alpha-melanocyte stimulating hormone as a potential immunosuppressive factor in aqueous humor. Curr. Eye Res. 11: 1199-1206.
B8. Taylor, A. W., J. W. Streilein & S. W. Cousins. 1994. Immunoreactive vasoactive intestinal peptide contributes to the immunosuppressive activity of normal aqueous humor. J. Immunol. 153: 1080-1086.
B10. Ferguson, T. A., S. Fletcher, J. Herndon & T. S. Griffith. 1995. Neuropeptides modulate immune deviation induced via the anterior chamber of the eye. J. Immunol. 155: 1746-1756.
B11. Taylor, A. W., J. W. Streilein & S. W. Cousins. 1994. Alpha-melanocyte-stimulating hormone suppresses antigen-stimulated T cell production of gamma-interferon. Neuroimmunomodulation 1: 188-194.
B12. Lee, T. H., A. B. Lerner & V. Buettner-Janusch. 1961. The isolation and structure of α- and β-melanocyte-stimulating hormones from monkey pituitary glands. J. Biol. Chem. 236: 1390-1394.
B13. Nakanishi, S., A. Inoue, T. Kita, M. Nakamura, A. C. Y. Chang, S. N. Cohen & S. Numa. 1979. Nucleotide sequence of cloned cDNA for bovine corticotropin-b-lipotropin precursor. Nature 278: 423-427.
B14. Lipton, J. M. 1990. Modulation of host defense by the neuropeptide α-MSH. Yale J. Bio. Med. 63: 173-182.
B15. Lipton, J. M. & A. Catania. 1997. Anti-inflammatory actions of the neuroimmunomodulator α-MSH. Immunol. Today 18: 140-145.
B16. Holdeman, M., O. Khorram, W. K. Samson & J. M. Lipton. 1985. Fever-specific changes in central MSH and CRF concentrations. Amer. J. Pysiol. 248: R125-R129.
B17. Watanabe, T., M. E. Hiltz, A. Catania & J. M. Lipton. 1993. Inhibition of IL-1β-induced periferal inflammation by peripheral and central administration of analogs of the neuropeptide α-MSH. Brain Res. Bull. 32: 311-314.
B18. Martin, L. W., A. Catania, M. E. Hiltz & J. M. Lipton. 1991. Neuropeptide alpha-MSH antagonizes IL-6- and TNF-induced fever. Peptides 12: 297-299.
B19. Chiao, H., S. Foster, R. Thomas, J. Lipton & R. A. Star. 1996. α-Melanocyte stimulating hormone reduces endotoxin-induced liver inflammation. J. Clin. Invest. 97: 2038-2044.
B20. Rajora, N., G. Ceriani, A. Catania, R. A. Star, M. T. Murphy & J. M. Lipton. 1996. α-MSH production, receptors, and influence on neopterin in a human monocyte/macrophage cell line. J Leuk. Biol. 59: 248-253.
B21. Star, R. A., N. Rajora, J. Huang, R. Chavez, A. Catania & J. M. Lipton. 1995. Evidence of autocrine modulation of macrophage nitric oxide synthase by α-MSH. Proc. Natl. Acad. Sci. USA 90: 8856-8860.
B22. Catania, A., N. Rajora, F. Capsoni, F. Minonzio, R. A. Star & J. M. Lipton. 1996. The neuropeptide α-MSH has specific receptors on neutrophils and reduces chemotaxis in vitro. Peptides 17: 675-679.
B23. Chakraborty, A. K., Y. Funasaka, A. Slominski, G. Ermak, J. Hwang, J. M. Pawelek & M. Ichihashi. 1996. Production and release of pro-opiomelanocortin (POMC) derived peptides by human melanocytes and keratinocytes in culture: regulation by ultraviolet B. Biochim. Biophys. Acta 1313: 130-138.
B24. O'Donohue, T. L. & D. M. Dorsa. 1982. The opiomelano-tropinergic neuronal and endocrine systems. Peptides 3: 353-395.
B26. Taylor, A. W., P. Alard, D. G. Yee & J. W. Streilein. 1997. Aqueous humor induces transforming growth factor-beta (TGF-beta)-producing regulatory T-cells. Curr. Eye Res. 16: 900-908.
B27. Silver, P. B., L. V. Rizzo, C. C. Chan, L. A. Donoso, B. Wiggert & R. R. Caspi. 1995. Identification of a major pathogenic epitope in the human IRBP molecule recognized by mice of the H-2r haplotype. Investi. Ophthalmol. Vis. Sci. 36: 946-954.
B28. Taylor, A. W. & J. W. Streilein. 1996. Inhibition of antigen-stimulated effector T cells by human cerebrospinal fluid. Neuroimmunomodulation 3: 112-118.
B29. Chen, Y., V. K. Kuchroo, J. I. Inobe, D. A. Hafler & H. L. Weiner. 1994. Regulatory T cell clones induced by oral tolerance: suppression of autoimmune encephalomyelitis. Science 265: 1237-1240.
B30. Caspi, R., L. Stiff, R. Morawentz, N. Miller-Rivero, C. Chan, B. Wiggert, R. Nussenblatt, H. Morse & L. Rizzo. 1996. Cytokine-dependent modulation of oral tolerance in a murine model of autoimmune uveitis. Ann. N.Y. Acade. Sci. 778: 315-324.
B31. Weiner, H. L. 1997. Oral tolerance: immune mechanisms and treatment of autoimmune diseases. Immunol. Today 18: 335-343.
B32. Shi, F. D., H. Li, H. Wang, X. Bai, P. H. van der Meide, H. Link & H. G. Ljunggren. 1999. Mechanisms of nasal tolerance induction in experimental autoimmune myasthenia gravis: identification of regulatory cells. J. Immunol. 162: 5757-5763.
B33. Inobe, J., A. J. Slavin, Y. Komagata, Y. Chen, L. Liu & H. L. Weiner. 1998. IL-4 is a differentiation factor for transforming growth factor-beta secreting Th3 cells and oral administration of IL-4 enhances oral tolerance in experimental allergic encephalomyelitis. Eur. J. Immunol. 28: 2780-2790.
B34. Takeuchi, M., M. M. Kosiewicz, P. Alard & J. W. Streilein. 1997. On the mechanisms by which transforming growth factor-β2 alters antigen-presenting abilities of macrophages on T cell activation. Eur. J. Immunol. 27: 1648-1656.
B35. Tsunawaki, S., M. Sporn, A. Ding & C. Nathan. 1988. Deactivation of macrophages by transforming growth factor-β. Nature 334: 260-262.
C1. Star R A, Rajora N, Huang J et al. Evidence of autocrine modulation of macrophage nitric oxide synthase by α-MSH. Proc. Natl. Acad. Sci. USA 1995; 90: 8856-60.
C2. Lee T H, Lerner A B, Buettner-Janusch V. The isolation and structure of α- and β-melanocyte-stimulating hormones from monkey pituitary glands. J. Biol. Chem. 1961; 236: 1390-4.
C3. Chakraborty A K, Funasaka Y, Slominski A et al. Production and release of proopiomelanocortin (POMC) derived peptides by human melanocytes and keratinocytes in culture: regulation by ultraviolet B. Biochim. Biophys. Acta 1996; 1313: 130-8.
C4. Rajora N, Ceriani G, Catania A et al. α-MSH production, receptors, and influence on neopterin in a human monocyte/macrophage cell line. JAMA 1996; 59: 248-53.
C5. Lipton J M, Catania A. Anti-inflammatory actions of the neuroimmunomodulator α-MSH. Immunol. Today 1997; 18: 140-5.
C6. Wintzen M, Gilchrest B A. Proopiomelanocortin, its derived peptides, and the skin. J. Invest. Derm. 1996; 106: 3-10.
C7. Learner A B, Wright M R. In vitro frog skin assay for agents that darken and lighten melanocytes. In: Methods of Biochemical Analysis (Glick D, ed), Vol. 8. New York, N.Y.: Interscience Publishers, Inc., 1960: 295-307.
C8. Manna S K, Aggarwal B B. α-Melanocyte stimulating hormone inhibits the nuclear transcription factor NF-κB activation induced by various infalmmatory agents. J. Immunol. 1998; 161: 2873-80.
C9. Ichiyama T, Sakai T, Catania A et al. Systemically administered α-melanocyte stimulating peptides inhibit NF-κB activation in experimental brain inflammation. Brain Res. 1999; 836: 31-7.
C10. Mason M J, Van Epps D. Modulation of IL-1, Tumor necrosis factor, and C5A-mediated murine neutrophil migration by α-melanocyte-stimulating hormone. J. Immunol. 1989; 142: 1646-51.
C11. Cannon J G, Tatro J B, Reichlin S et al. α Melanocyte stimulating hormone inhibits immunostimulatory and inflammatory actions of interleukin 1. J. Immunol. 1986; 137: 2232-6.
C12. Catania A, Rajora N, Capsoni F et al. The neuropeptide α-MSH has specific receptors on neutrophils and reduces chemotaxis in vito. Peptides 1996; 17: 675-9.
C13. Martin L W, Lipton J M. Acute phase response to endotoxin: rise in plasma α-MSH and effects of α-MSH injection. Amer. J. Pysiol. 1990; 259: R768-72.
C14. Holdeman M, Khorram O, Samson W K et al. Fever-specific changes in central MSH and CRF concentrations. Amer. J. Pysiol. 1985; 248: R125-R9.
C15. Watanabe T, Hiltz M E, Catania A et al. Inhibition of IL-1β-induced periferal inflammation by peripheral and central administration of analogs of the neuropeptide α-MSH. Brain Res. Bull. 1993; 32: 311-4.
C16. Hiltz M E, Catania A, Lipton J M. Alpha-MSH peptides inhibit acute inflammation induced in mice by rIL-1 beta, rIL-6, rTNF-alpha and endogenous pyrogen but not that caused by LTB4, PAF and rIL-8. Cytokine 1992; 4: 320-8.
C17. Shih S T, Khorram O, Lipton J M et al. Central administration of α-MSH antiserum augments fever in the rabbit. Am. J. Physiol. 1986; 250: R803-R6.
C18. Chiao H, Foster S, Thomas R et al. α-Melanocyte stimulating hormone reduces endotoxin-induced liver inflammation. J. Clin. Invest. 1996; 97: 2038-44.
C19. Martin L W, Catania A, Hiltz M E et al. Neuropeptide alpha-MSH antagonizes IL-6- and TNF-induced fever. Peptides 1991; 12: 297-9.
C20. Lipton J M. Modulation of host defense by the neuropeptide α-MSH. Yale J. Bio. Med. 1990; 63: 173-82.
C21. Grabbe S, Bhardwaj R S, Mahnke K et al. α-Melanocyte stimulating hormone induces hapten-specific tolerance in mice. J. Immunol. 1996; 156: 473-8.
C22. Bhardwaj R S, Schwarz A, Becher E et al. Pro-opiomelanocortin-derived peptides induce IL-10 production in human monocytes. J. Immunol. 1996; 156: 2517-21.
C23. Taylor A W, Streilein J W, Cousins S W. Identification of alpha-melanocyte stimulating hormone as a potential immunosuppressive factor in aqueous humor. Curr. Eye Res. 1992; 11: 1199-206.
C24. Taylor A W, Streilein J W, Cousins S W. Alpha-melanocyte-stimulating hormone suppresses antigen-stimulated T cell production of gamma-interferon. Neuroimmunomodulation 1994; 1: 188-94.
C25. Taylor A W. Ocular immunosuppressive microenvironment. Chem. Immunol. 1999; 73: 72-89.
C26. Taylor A W, Alard P, Yee D G et al. Aqueous humor induces transforming growth factor-β (TGF-β)-producing regulatory T-cells. Curr. Eye Res. 1997; 16: 900-8.
C27. Caspi R, Roberge F, Chan C et al. A new model of autoimmune disease, experimental autoimmune uveoretinitis induced in mice with two different retinal antigens. J. Immunol. 1988; 140: 1490-5.
C28. Taylor A W, Yee DG, Nishida T et al. Neuropeptide regulation of immunity; the immunosuppressive activity of alpha-melanocyte stimulating hormone (α-MSH). Ann. N.Y. Acade. Sci. 2000; 917: 239-47.
C29. Nishida T, Taylor A W. Specific aqueous humor factors induce activation of regulatory T cells. Investi. Ophthalmol. Vis. Sci. 1999; 40: 2268-74.
C30. Silver P, Rizzo L, Chan C et al. Identification of a major pathogenic epitope in the human IRBP molecule recognized by mice of the H-2^{r} haplotype. Investi. Ophthalmol. Vis. Sci. 1995; 36: 946-54.
C31. Weiner H L. Oral tolerance: immune mechanisms and treatment of autoimmune diseases. Immunol. Today 1997; 18: 335-43.
C32. Mountjoy K G, Robbins L S, Mortrud M T et al. The cloning of a family of genes that encode the melanocortain receptors. Science 1992; 257: 1248-51.
C33. Buggy J J. Binding of α-melanocyte-stimulating hormone to its G-protein-coupled receptor on B-lymphocytes activates the Jak/STAT pathway. Biochem. J. 1998; 331: 211-6.
C34. Clarke B L. Binding and processing of 1-ACTH by isolated rat splenic lymphocytes. Bioch. Biophy. Res. Comm. 1999; 266: 542-6.
C35. Shevach E M, Thornton A, Suri-Payer E. T lymphocyte-mediated control of autoimmunity. Novartis Found Symp 1998; 215: 200-11; discussion 11-30.
C36. Thornton A M, Shevach E M. CD4+CD25+ immunoregulatory T cells suppress polyclonal T cell activation in vitro by inhibiting interleukin 2 production. J. Exp. Med. 1998; 188: 287-96.
C37. Suri-Payer E, Amar A Z, Thornton A M et al. CD4+CD25+ T cells inhibit both the induction and effector function of autoreactive T cells and represent a unique linage of immunoregulatory cells. J. Immunol. 1998; 160: 1212-8.
C38. Takahashi T, Tagami T, Yamazaki S et al. Immunologic self-tolerance maintained by CD25(+)CD4(+) regulatory T cells constitutively expressing cytotoxic T lymphocyte-associated antigen 4. J. Exp. Med. 2000; 192: 303-10.
C39. Read S, Malmstrom V, Powrie F. Cytotoxic T lymphocyte-associated antigen 4 plays an essential role in the function of CD25(+)CD4(+) regulatory cells that control intestinal inflammation. J. Exp. Med. 2000; 192: 295-302.
C40. Salomon B, Lenschow D J, Rhee L et al. B7/CD28 costimulation is essential for the homeostasis of the CD4+CD25+ immunoregulatory T cells that control autoimmune diabetes. Immunity 2000; 12: 431-40.
C41. Read S, Mauze S, Asseman C et al. CD38+ CD45RB(low) CD4+ T cells: a population of T cells with immune regulatory activities in vitro. Eur. J. Immunol. 1998; 28: 3435-47.
C42. Seddon B, Mason D. Peripheral autoantigen induces regulatory T cells that prevent autoimmunity. J. Exp. Med. 1999; 189: 877-82.
C43. Chen Y, Kuchroo V K, Inobe J I et al. Regulatory T cell clones induced by oral tolerance: suppression of autoimmune encephalomyelitis. Science 1994; 265: 1237-40.
C44. Caspi R R, Stiff L R, Morawentz R et al. Cytokine-dependent modulation of oral tolerance in a murine model of autoimmune uveitis. Ann. N.Y. Acade. Sci. 1996; 778: 315-24.
C45. Maggi E, Parronchi P, Manetti R et al. Reciprocal regulatory effects of INF-g and IL-4 on the in vitro development of human Th1 and Th2 clones. J. Immunol. 1992; 148: 2142-7.
C46. Mountjoy K G, Mortrud M T, Low M J et al. Localization of the melanocortin-4 receptor (MC4-R) in neuroendocrine and autonomic control circuts in the brain. Mol. Endocrinol. 1994; 8: 1298-308.
C47. Huszar D, Lynch C A, Fairchild-Huntress V et al. Targeted disruption of the melanocortin-4 receptor results in obesity in mice. Cell 1997; 88: 131-41.
C48. Schioth H B, Mutulis F, Muceniece R et al. Selective properties of C- and N-terminals and core residues of the melanocyte-stimulating hormone on binding to the human melanocortin receptor subtypes. Eur J Pharmacol 1998; 349: 359-66.

### EQUIVALENTS

While the present invention has been described in conjunction with certain preferred embodiments, one of ordinary skill in the art, after reading the foregoing specification, will be able to effect various changes, substitutions of equivalents, and other alterations to the compositions and methods set forth herein, without departing from the spirit of the invention. It is therefore intended that the protection granted by Letters Patent hereon be limited only by the definitions contained in the appended claims and equivalents thereof.

What is claimed is:

1. A method for generating antigen-specific, regulatory CD4+/CD25+ T cells that produce Transforming Growth Factor β (TGF-β), comprising:
exposing CD3-enriched, primed T cells to a specific antigen in the presence of antigen-presenting cells and a composition comprising an effective amount of alpha-Melanocyte Stimulating Hormone (α-MSH) or an analogue or derivative of α-MSH comprising an α-MSH receptor-binding portion thereof, wherein the specific antigen is an antigen recognized by the primed T cells.

2. A method for generating antigen-specific, regulatory CD4+/CD25+ T cells that produce Transforming Growth Factor β (TGF-β), comprising:
exposing CD3-enriched, primed T cells to a T cell receptor (TCR)-crosslinking agent in the presence of an effective amount of α-MSH or an analogue or derivative of α-MSH comprising an α-MSH receptor-binding portion thereof.

3. The method of claim 1 or 2, further comprising, approximately 4-6 hours after said first exposure step has begun, additionally exposing the primed T cells to an effective amount of Transforming Growth Factor-β2 (TGF-β2).

4. The method of claim 3, wherein the exposure to TGF-β2 is achieved by including in the composition, an effective amount of TGF-β2 in an timed-release delivery vehicle.

5. The method of claim 1 or 2, wherein the exposing step is performed in vitro under T cell culture conditions.

6. The method of claim 1, wherein the exposing step is performed in vivo in an animal.

7. A method for down-regulating an autoimmune response or other T cell-mediated inflammatory response, comprising:
(a) harvesting T cells from the animal;
(b) inducing TGF-β-producing, regulatory T cells by exposing the harvested T cells in vitro to a specific antigen under culture conditions enabling stimulation of at least one primed memory T cell that specifically recognizes said antigen;
(c) exposing the primed T cells in vitro to a specific antigen in the presence of a composition comprising an effective amount of alpha-Melanocyte Stimulating Hormone (α-MSH) or an analogue or derivative of α-MSH comprising an α-MSH receptor-binding portion thereof, and in the presence of at least one T cell receptor (TCR)-crosslinking agent, under T cell culture conditions; and
(d) injecting into an animal, primed T cells treated in accordance with step (c).

8. The method of claim 7, wherein step (c) further comprises the addition of an effective amount of TGF-β2, approximately 4-6 hours after the start of the exposure of the primed T cells to the specific antigen and the α-MSH.

9. The method of claim 7 or 8, wherein, between steps (c) and (d), the primed T cells treated in accordance with step (c) are enriched for CD4+/CD25+, TGF-β-producing T cells.

10. The method of claim 7 or 8, wherein the TCR-crosslinking agent is an anti-CD3 monoclonal antibody.

11. The method of claim 7 or 8, wherein the TCR-crosslinking agent is a T cell mitogen selected from the group consisting of: concanavalin-A (ConA); phytohemagglutinin (PHA); and pokeweed mitogen (PWM).

12. The method of claim 1, 2, or 7, wherein the effective amount of α-MSH or an analogue or derivative of α-MSH comprising an α-MSH receptor-binding portion thereof, is an amount sufficient to produce an in situ concentration of at least approximately 30 pg/ml of whole α-MSH or an analogue or derivative of α-MSH comprising a molar equivalent amount of an α-MSH receptor-binding portion thereof, in the immediate vicinity of the primed T cells during the exposing step.

13. The method of claim 1, 2, or 7, wherein the effective amount of α-MSH or an analogue or derivative of α-MSH comprising an α-MSH receptor-binding portion thereof, is an amount sufficient to produce an in situ concentration in the range of approximately 30-100 pg/ml in the immediate vicinity of the primed T cells during the exposing step.

14. The method of claim 3 or 8, wherein the effective amount of TGF-β2 is an amount sufficient to produce an in situ TGF-β2 concentration that lies within the range of approximately 1-10 ng/ml in the immediate vicinity of the primed T cells during the exposing step.

15. The method of claim 3 or 8, wherein the effective amount of TGF-β2 is an amount sufficient to produce an in situ TGF-β2 concentration of approximately 5.0 ng/ml in the immediate vicinity of the primed T cells during the exposing step.

16. The method of claim 1, 2, 7 or 8, wherein the exposing step comprises incubating the T cells in vitro with the specific antigen and the composition at approximately 37° C., for a period within the range of approximately 18-24 hours, in substantially serum-free T cell culture conditions.

17. The method of claim 16, wherein the substantially serum-free T cell medium includes RPMI 1640, an approximately 500-fold dilution of ITS+ solution and approximately 0.1% bovine serum albumin.

18. The method of claim 5, 7, 23, 24, or 25, wherein the animal is a human, a mouse, a rat, a dog, a cat, a rabbit, or a horse.

19. A kit for generating antigen-specific regulatory T cells, comprising:
(a) a specific antigen;
(b) α-MSH or an analogue or derivative of α-MSH comprising an α-MSH receptor-binding portion thereof; and
(c) an article of manufacture comprising instructions on how to use components (a) and (b) to generate TGF-β-producing, CD4+/CD25+, regulatory T cells.

20. The kit of claim 19, further comprising: (d) TGF-β2, and wherein the article of manufacture further comprises instructions for using the TGF-β2.

21. The kit of claim 19, wherein the specific antigen comprises a target molecule of an autoimmune disorder.

22. The kit of claim 21, wherein the target molecule is selected from the group consisting of: a glycoprotein; a protein; a polypeptide; a synthetic amino acid polypeptide; a recombinant amino acid polypeptide; a carbohydrate moiety; an oligonucleotide; a DNA; a RNA; and a whole microorganism.

23. A method for down-regulating a graft rejection response in a graft recipient, comprising:
(a) transfecting a graft tissue or organ with genetic material for expressing α-MSH or an analogue or derivative of α-MSH comprising an α-MSH receptor binding portion thereof in said graft; and
(b) implanting the transfected graft from step (a) into a recipient animal.

24. A method for down-regulating a T cell-mediated autoimmune response in a tissue site in an animal, comprising directly injecting genetic material for expressing α-MSH, into or near the autoimmune-diseased tissue site.

25. A method for down-regulating a T-cell-mediated autoimmune response in a tissue site in an animal, comprising:
(a) harvesting a tissue sample from the tissue site;
(b) transfecting the harvested tissue sample with genetic material for expressing α-MSH or an analogue or derivative of α-MSH comprising an α-MSH receptor-binding portion thereof; and
(c) implanting the transfected tissue sample into the animal.

26. A method of suppressing a T cell-mediated autoimmune graft rejection response in an animal, comprising:
(a) systemically injecting into the animal, an effective amount of α-MSH or an analogue or derivative of α-MSH comprising an α-MSH receptor-binding portion thereof; and
(b) measuring the peripheral level of CD4+/CD25+ T cells in said animal.

27. The method of claim 26, wherein the effective amount of α-MSH or an analogue or derivative of α-MSH comprising an α-MSH receptor-binding portion thereof, is an amount sufficient to produce a peripheral blood concentration of at least approximately 30 pg/ml of whole α-MSH or a molar equivalent concentration of an α-MSH receptor-binding portion of α-MSH.

28. A method of down-regulating or suppressing an autoimmune disorder or a graft rejection response in an animal by transfecting a cell within the animal with genetic material coding for an antigen that also comprises lysine-proline-valine.

29. The method of claim 23, 25, or 28, wherein the transfecting step is performed using an episomal transfection technique.

30. The method of claim 23, 25, or 28, wherein the transfecting step is performed using a chromosomal transfection technique.

31. A method of regulating a T cell-mediated immune response in a mammal, said method comprising the steps of:
(a) providing a mammal; and
(b) administering to said mammal an effective amount of α-MSH or an analogue or a derivative of α-MSH, said analogue or derivative having α-MSH functional activity, wherein said α-MSH functional activity is mediated exclusively through melanocortin 5 receptor (MC5r),
wherein said step of administering regulates said T cell-mediated immune response.

32. The method of claim 31, wherein said α-MSH is a synthetic analogue wherein said analogue mediates the activation of regulatory T cells.

33. The method of claim 31, wherein said α-MSH is attached to a polyclonal or monoclonal antibody, wherein said antibody acts as an agonist to the bound MC5r receptor.

34. The method of claim 33 wherein said antibody is an anti-MC5r antibody, or fragment or derivative thereof.

35. The method of claim 34 wherein said anti-MC5r antibody is an anti-MC5r antibody F(ab)₂ fragment.

36. The method of claim 31, wherein said regulation of T cell-mediated immune response is suppression of T cell-mediated inflammatory response.

37. The method of claim 31, wherein said regulation of T cell-mediated immune response is induction of CD4⁺/CD25⁺ regulatory T cells that produces TGF-β.

38. A method for down-regulating a T cell-mediated autoimmune response in autoimmune disease tissue site in an animal, comprising directly injecting α-MSH or an analogue or derivative of α-MSH comprising an α-MSH receptor-binding portion thereof, into or near the autoimmune-diseased tissue site.

39. A method for down-regulating a T-cell-mediated autoimmune response in a tissue site in an animal, comprising:
(a) harvesting a tissue sample from the tissue site;
(b) treating the harvested tissue sample with α-MSH or an analogue or derivative of α-MSH comprising an α-MSH receptor-binding portion thereof; and
(c) implanting the treated tissue sample into the animal.

40. A method for down-regulating a T cell-mediated autoimmune response in autoimmune disease tissue site in an animal, said method comprising the steps of:
(a) providing said animal; and
(b) directly injecting an effective amount of α-MSH or an analogue or derivative of α-MSH comprising an α-MSH receptor-binding portion thereof, into or near the autoimmune-diseased tissue site in an animal; wherein said effective amount is an amount sufficient to produce an in situ concentration in the range of about 30-100 pg/ml.

41. A method for down-regulating a T-cell-mediated autoimmune response in a tissue site in an animal, comprising:
(a) harvesting a tissue sample from the tissue site;
(b) treating the harvested tissue sample with an effective amount of α-MSH or an analogue or derivative of α-MSH comprising an α-MSH receptor-binding portion thereof;
wherein said effective amount is an amount sufficient to produce an in situ concentration in the range of about 30-100 pg/ml; and
(c) implanting the treated tissue sample into the animal.

42. The method of claim 24, wherein the animal is a human, a mouse, a rat, a dog, a cat, a rabbit or a horse.

43. The method of claim 24, wherein the autoimmune-diseased tissue site is an eye of the animal.
